(19) United States
(12) Patent Application Publication
(10) Pub. No.: US 2022/0319639 A1
(43) Pub. Date: Oct. 6, 2022

(54) ARTIFICIAL INTELLIGENCE-BASED BASE CALLER WITH CONTEXTUAL AWARENESS

(71) Applicant: Illumina, Inc., San Diego, CA (US)

(72) Inventor: Amirali KIA, San Mateo, CA (US)

(73) Assignee: Illumina, Inc., San Diego, CA (US)

(21) Appl. No.: 17/687,586

(22) Filed: Mar. 4, 2022

Related U.S. Application Data
(60) Provisional application No. 63/169,163, filed on Mar. 31, 2021.

(51) Int. Cl. G16B 30/00; G16B 40/00; G06N 3/04; G06T 7/00

(52) U.S. Cl. CPC G16B 30/00; G16B 40/00; G06N 3/04; G06T 7/0012

(57) ABSTRACT

A neural network processes sequencing images on a patch-by-patch basis for base calling. The sequencing images depict intensity emissions of a set of analytes. The patches depict the intensity emissions for a subset of the analytes and have undiverse intensity patterns due to limited base diversity. The neural network has convolution filters that have receptive fields confined to the patches. The convolution filters detect intensity patterns in the patches with losses in detection due to the undiverse intensity patterns and confined receptive fields. An intensity contextualization unit determines intensity context data based on intensity values in the images. The data flow logic appends the intensity context data to the sequencing images to generate intensity contextualized images. The neural network applies the convolution filters on the intensity contextualized images and generates base call classifications. The intensity context data in the intensity contextualized images compensates for the losses in detection.

### PRIORITY APPLICATION

This application claims priority to or the benefit of U.S. Provisional Patent Application No. 63/169,163, titled, “Artificial Intelligence-Based Base Caller with Contextual Awareness,” by Amirali Kia filed Mar. 31, 2021 (Attorney Docket No. ILLM 1033-1/IP-2007-PRV).

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using neural networks such as convolutional neural networks for analyzing data.

### INCORPORATIONS

The following are incorporated by reference for all purposes as if fully set forth herein: U.S. Patent Application No. 62/979,384, titled “ARTIFICIAL INTELLIGENCE-BASED BASE CALLING OF INDEX SEQUENCES,” filed 20 Feb. 2020 (Attorney Docket No. ILLM 1015-1/IP-1857-PRV);

U.S. Patent Application No. 62/979,414, titled “ARTIFICIAL INTELLIGENCE-BASED MANY-TO-MANY BASE CALLING,” filed 20 Feb. 2020 (Attorney Docket No. ILLM 1016-1/IP-1858-PRV);

U.S. Patent Application No. 62/979,385, titled “KNOWLEDGE DISTILLATION-BASED COMPRESSION OF ARTIFICIAL INTELLIGENCE-BASED BASE CALLER,” filed 20 Feb. 2020 (Attorney Docket No. ILLM 1017-1/IP-1859-PRV);

U.S. Patent Application No. 63/072,032, titled “DEFECTING AND FILTERING CLUSTERS BASED ON ARTIFICIAL INTELLIGENCE-PREDICTED BASE CALLS,” filed 28 Aug. 2020 (Attorney Docket No. ILLM 1018-1/IP-1860-PRV);

U.S. Patent Application No. 62/979,412, titled “MULTI-CYCLE CLUS IER BASED REAL TIME ANALYSIS SYSTEM,” filed 20 Feb. 2020 (Attorney Docket No. ILLM 1020-1/IP-1866-PRV);

U.S. Patent Application No. 62/979,411, titled “DATA COMPRESSION FOR ARTIFICIAL INTELLIGENCE-BASED BASE CALLING,” filed 20 Feb. 2020 (Attorney Docket No. ILLM 1029-1/IP-1964-PRV);

U.S. patent application Ser. No. 17/179,395, titled “DATA COMPRESSION FOR ARTIFICIAL INTELLIGENCE-BASED BASE CALLING,” filed 18 Feb. 2021 (Attorney Docket No. ILLM 1029-2/IP-1964-US);

U.S. Patent Application No. 62/979,399, titled “SQUEEZING LAYER FOR ARTIFICIAL INTELLIGENCE-BASED BASE CALLING,” filed 20 Feb. 2020 (Attorney Docket No. ILLM 1030-1/IP-1982-PRV);

U.S. patent application Ser. No. 17/180,480, titled “SPLIT ARCHITECTURE FOR ARTIFICIAL INTELLIGENCE-BASED BASE CALLER,” filed 19 Feb. 2021 (Attorney Docket No. ILLM 1030-2/IP-1982-US);

U.S. patent application Ser. No. 17/180,513, titled “BUS NETWORK FOR ARTIFICIAL INTELLIGENCE-BASED BASE CALLER,” filed 19 Feb. 2021 (Attorney Docket No. ILLM 1031-2/IP-1965-US);

U.S. patent application Ser. No. 16/825,987, titled “TRAINING DATA GENERATION FOR ARTIFICIAL INTELLIGENCE-BASED SEQUENCING,” filed 20 Mar. 2020 (Attorney Docket No. ILLM 1008-16/IP-1693-US);

U.S. patent application Ser. No. 16/825,991 titled “ARTIFICIAL INTELLIGENCE-BASED GENERATION OF SEQUENCING METADATA,” filed 20 Mar. 2020 (Attorney Docket No. ILLM 1008-17/IP-1741-US);

U.S. patent application Ser. No. 16/826,126, titled “ARTIFICIAL INTELLIGENCE-BASED BASE CALLING,” filed 20 Mar. 2020 (Attorney Docket No. ILLM 1008-18/IP-1744-US);

U.S. patent application Ser. No. 16/826,134, titled “ARTIFICIAL INTELLIGENCE-BASED QUALITY SCORING,” filed 20 Mar. 2020 (Attorney Docket No. ILLM 1008-19/IP-1747-US); and

U.S. patent application Ser. No. 16/826,168, titled “ARTIFICIAL INTELLIGENCE-BASED SEQUENCING,” filed 21 Mar. 2020 (Attorney Docket No. ILLM 1008-20/IP-1752-PRV-US).

### BACKGROUND

The subject matter discussed in this section should not be assumed to be prior art merely because of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Convolutional neural networks are the current state-of-the-art machine learning algorithms for many tasks in computer vision, such as classification or segmentation. Training convolutional neural networks requires large amounts of computer memory, which increases exponentially with increasing image size. Computer memory becomes a limiting factor because the backpropagation algorithm for optimizing deep neural networks requires the storage of intermediate activations. Since the size of these intermediate activations in the convolutional neural networks increases proportionate to the input size, memory quickly fills up with large images.

The problem of large images is circumvented by downsampling the original image or processing the original image on a patch-by-patch basis. Both approaches have significant drawbacks: the former results in a loss of local details, whereas the latter results in losing global contextual information. The receptive field of convolution filters of the convolutional neural networks is at most the size of the patches. The convolution filters disregard spatial relationships between the patches, limiting the incorporation of contextual information from outside a subject patch.

Therefore, an opportunity arises to improve base calling by analyzing both local details in a patch and global context outside the patch. More accurate base calling with reduced error rates may result.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The color drawings also may be available in PAIR via the Supplemental Content tab. In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 is a simplified block diagram that shows various aspects of the technology disclosed.

FIG. 2 illustrates one implementation of accessing a sequencing image on a patch-by-patch basis for base calling.

FIG. 3 shows one implementation of generating intensity contextualized images.

FIG. 4 shows one example of a full image from which a patch is accessed by the neural network such that the patch is centered at a target cluster to be base called.

FIG. 5 depicts one implementation of the intensity contextualization unit having a plurality of convolution pipelines.

FIG. 6 illustrates one implementation of the neural network processing an intensity contextualized patch and generating the base calls.

FIG. 7 shows one implementation of the neural network processing previous, current, and successive intensity contextualized images for a plurality of sequencing cycles and generating the base calls.

FIG. 8 demonstrates base calling superiority of the disclosed neural network-based base caller configured with the disclosed intensity contextualization unit over another neural network-based base caller (DeepRTA) and another non-neural network-based base caller (RTA).

FIG. 9 shows the base calling error rates observed for various combinations (configurations) of filter sizes (or kernel sizes), strides, and filter bank sizes (K) of convolution filters of the disclosed neural network-based base caller.

FIG. 10 compares base calling error rate of DeepRTA against base calling error rates of different filter bank size configurations (K0s) of the disclosed neural network-based base caller configured with the disclosed intensity contextualization unit (DeepRTA-K0-04; DeepRTA-K0-06; DeepRTA-K0-10; DeepRTA-K0-16; DeepRTA-K0-18; and DeepRTA-K0-20).

FIG. 11 shows base calling error rates when the disclosed neural network-based base caller configured with the disclosed intensity contextualization unit extracts intensity context data from an original input image of size 115×115 (red fitted line) versus an original input image of size 160×160 (blue fitted line).

FIG. 12 shows base calling accuracy (1-base calling error rate) of the different configurations of the disclosed neural network-based base caller configured with the disclosed intensity contextualization unit, i.e., DeepRTA-K0-06, DeepRTA-349-K0-10-160p, DeepRTA-K0-16, DeepRTA-K0-16-Lanczos, DeepRTA-K0-18, and DeepRTA-K0-20 against DeepRTA over base calling homopolymers (e.g., GGGGG) and flanked-homopolymers (e.g., GGTGG).

FIG. 13 compares base calling error rates of the disclosed neural network-based base caller configured with the disclosed intensity contextualization unit and trained on normalized sequencing images (“DeepRTA-V2:349”) against DeepRTA, RTA, the disclosed neural network-based base caller configured with the disclosed intensity contextualization unit, trained on, and performing inference on normalized sequencing images (“DeepRTA-V2:349”), and the DeepRTA trained on and performing inference on normalized sequencing images (“DeepRTA-norm”).

FIGS. 14A and 14B depict one implementation of a sequencing system. The sequencing system comprises a configurable processor.

FIG. 14C is a simplified block diagram of a system for analysis of sensor data from the sequencing system, such as base call sensor outputs.

FIG. 15 is a simplified diagram showing aspects of the base calling operation, including functions of a runtime program executed by a host processor.

FIG. 16 is a simplified diagram of a configuration of a configurable processor such as the one depicted in FIG. 14C.

FIG. 17 is a computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

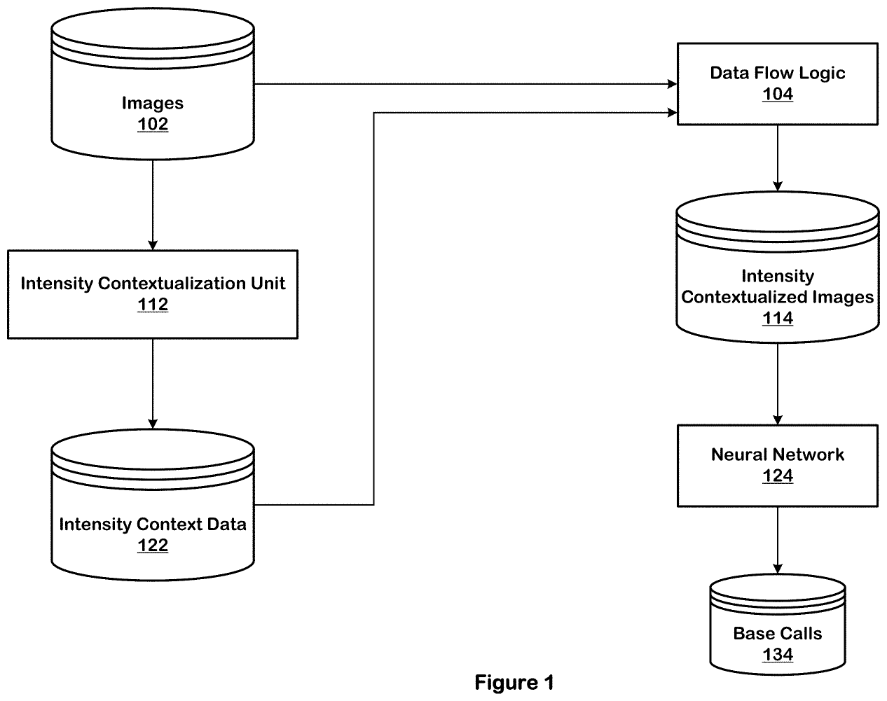

The technology disclosed provides an artificial intelligence-based base caller with contextual awareness. FIG. 1 is a simplified block diagram that shows various aspects of the technology disclosed. FIG. 1 includes images 102, a data flow logic 104, an intensity contextualization unit 112 (also referred to herein as “patch processing unit (PPU)”), intensity context data 122, intensity contextualized images 114, a neural network 124 (or neural network-based base caller), and base calls 134. The system can be formed by one or more programmed computers, with programming being stored on one or more machine readable media with code executed to carry out one or more steps of methods described herein. In the illustrated implementation, for example, the system includes the data flow logic 104 configured to output the intensity contextualized images 114 as digital image data, for example, image data that is representative of individual picture elements or pixels that, together, form an image of an array or other object.

### Sequencing Images

Base calling is the process of determining the nucleotide composition of a sequence. Base calling involves analyzing image data, i.e., sequencing images, produced during a sequencing run (or sequencing reaction) carried out by a sequencing instrument such as Illumina's iSeq, HiSeqX, HiSeq 3000, HiSeq 4000, HiSeq 2500, NovaSeq 6000, NextSeq 550, NextSeq 1000, NextSeq 2000, NextSeqDx, MiSeq, and MiSeqDx.

The following discussion outlines how the sequencing images are generated and what they depict, in accordance with one implementation.

Base calling decodes the intensity data encoded in the sequencing images into nucleotide sequences. In one implementation, the Illumina sequencing platforms employ cyclic reversible termination (CRT) chemistry for base calling. The process relies on growing nascent strands complementary to template strands with fluorescently-labeled nucleotides, while tracking the emitted signal of each newly added nucleotide. The fluorescently-labeled nucleotides have a 3′ removable block that anchors a fluorophore signal of the nucleotide type.

Sequencing occurs in repetitive cycles, each comprising three steps: (a) extension of a nascent strand by adding the fluorescently-labeled nucleotide; (b) excitation of the fluorophore using one or more lasers of an optical system of the sequencing instrument and imaging through different filters of the optical system, yielding the sequencing images; and (c) cleavage of the fluorophore and removal of the 3′ block in preparation for the next sequencing cycle. Incorporation and imaging cycles are repeated up to a designated number of sequencing cycles, defining the read length. Using this approach, each cycle interrogates a new position along the template strands.

The tremendous power of the Illumina sequencers stems from their ability to simultaneously execute and sense millions or even billions of clusters (also called “analytes”) undergoing CRT reactions. A cluster comprises approximately one thousand identical copies of a template strand, though clusters vary in size and shape. The clusters are grown from the template strand, prior to the sequencing run, by bridge amplification or exclusion amplification of the input library. The purpose of the amplification and cluster growth is to increase the intensity of the emitted signal since the imaging device cannot reliably sense fluorophore signal of a single strand. However, the physical distance of the strands within a cluster is small, so the imaging device perceives the cluster of strands as a single spot.

Sequencing occurs in a flow cell (or biosensor)—a small glass slide that holds the input strands. The flow cell is connected to the optical system, which comprises microscopic imaging, excitation lasers, and fluorescence filters. The flow cell comprises multiple chambers called lanes. The lanes are physically separated from each other and may contain different tagged sequencing libraries, distinguishable without sample cross contamination. In some implementations, the flow cell comprises a patterned surface. A “patterned surface” refers to an arrangement of different regions in or on an exposed layer of a solid support.

The imaging device of the sequencing instrument (e.g., a solid-state imager such as a charge-coupled device (CCD) or a complementary metal—oxide—semiconductor (CMOS) sensor) takes snapshots at multiple locations along the lanes in a series of non-overlapping regions called tiles. For example, there can be sixty four or ninety six tiles per lane. A tile holds hundreds of thousands to millions of clusters.

The output of the sequencing run is the sequencing images. Sequencing images depict intensity emissions of the clusters and their surrounding background using a grid (or array) of pixelated units (e.g., pixels, superpixels, subpixels). The intensity emissions are stored as intensity values of the pixelated units. The sequencing images have dimensions w×h of the grid of pixelated units, where w (width) and h (height) are any numbers ranging from 1 and 100,000 (e.g., 115×115, 200×200, 1800×2000, 2200×25000, 2800×3600, 4000×400). In some implementations, w and h are the same. In other implementations, w and h are different. The sequencing images depict intensity emissions generated as a result of nucleotide incorporation in the nucleotide sequences during the sequencing run. The intensity emissions are from associated clusters and their surrounding background.

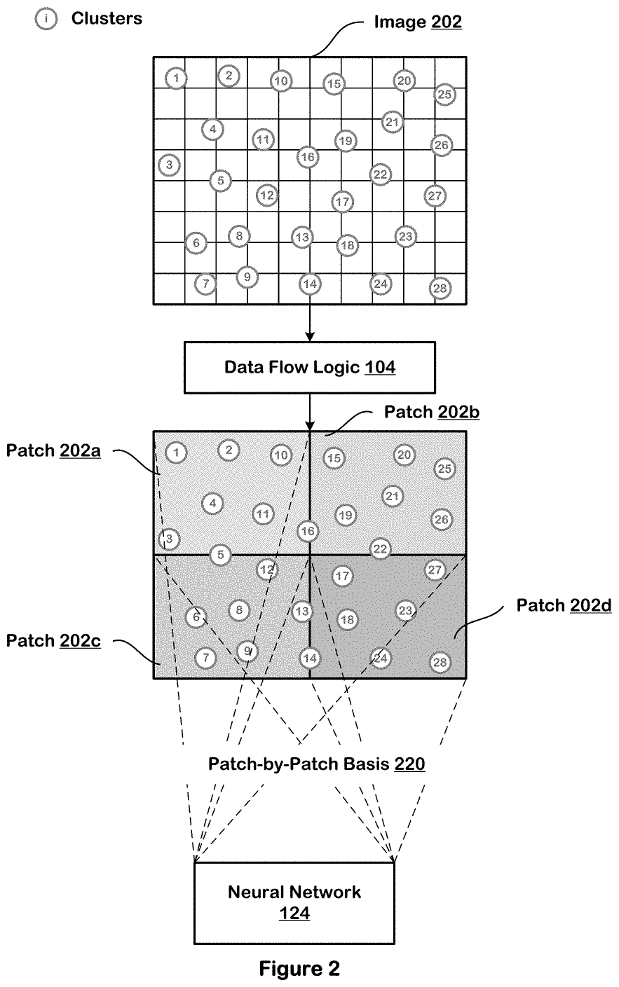

FIG. 2 illustrates one implementation of accessing a sequencing image 202 on a patch-by-patch basis 220 for base calling. In the illustrated example, the data flow logic 104 provides the sequencing image 202 to the neural network 124 for base calling. The neural network 124 accesses the sequencing image 202 on the patch-by-patch basis 220, for example, patches 202a, 202b, 202c, and 202d. Each of the patches is a sub-grid (or sub-array) of pixelated units in the grid of pixelated units that forms the sequencing image 202. The patches have dimensions q×r of the sub-grid of pixelated units, where q (width) and r (height) can be, for example, 1×1, 3×3, 5×5, 7×7, 10×10, 15×15, 25×25, and so on. In some implementations, q and r are the same. In other implementations, q and r are different. In some implementations, the patches are of the same size. In other implementations, the patches are of different sizes. In some implementations, the patches can have overlapping pixelated units (e.g., on the edges).

In the illustrated example, the sequencing image 202 depicts intensity emissions of a set of twenty-eight clusters 1-28. The patches depict the intensity emissions for a subset of the clusters. For example, the patch 202a substantially depicts the intensity emissions for a first subset of seven clusters 1, 2, 3, 4, 5, 10, and 16; the patch 202b substantially depicts the intensity emissions for a second subset of eight clusters 15, 16, 19, 20, 21, 22, 25, and 26; the patch 202c substantially depicts the intensity emissions for a third subset of eight clusters 5, 6, 7, 8, 9, 12, 13, and 14; and the patch 202d substantially depicts the intensity emissions for a fourth subset of nine clusters 13, 14, 17, 18, 22, 23, 24, 27, and 28.

Sequencing produces m sequencing image(s) per sequencing cycle for corresponding m image channel(s). That is, each of the images 102 has one or more image (or intensity) channels (analogous to the red, green, blue (RGB) channels of a color image). In one implementation, each image channel corresponds to one of a plurality of filter wavelength bands. In another implementation, each image channel corresponds to one of a plurality of imaging events at a sequencing cycle. In yet another implementation, each image channel corresponds to a combination of illumination with a specific laser and imaging through a specific optical filter. The patches are accessed from each of the m image channel(s) for a particular sequencing cycle. In different implementations such as 4-, 2-, and 1-channel chemistries, m is 4 or 2. In other implementations, m is 1, 3, or greater than 4.

Consider, for example, that the sequencing uses two different image channels: a blue channel and a green channel. Then, at each sequencing cycle, the sequencing produces a blue image and a green image. This way, for a series of k sequencing cycles, a sequence with k pairs of blue and green images is produced as output and stored as the images 102. Accordingly, a sequence of per-cycle image patches is generated for a series of k sequencing cycles of a sequencing run. The per-cycle image patches contain intensity data for associated clusters and their surrounding background in one or more image channels (e.g., a red channel and a green channel). In one implementation, when a single target cluster (e.g., cluster) is to be base called, the per-cycle image patches are centered at a center pixel that contains intensity data for a target associated cluster and non-center pixels in the per-cycle image patches contain intensity data for associated clusters adjacent to the target associated cluster.

The patches have undiverse (indistinguishable) intensity patterns due to limited base diversity of clusters in the subset. Compared to a full image, a patch is smaller and has fewer clusters, which in turn reduces the base diversity. The patch has scarce base variety because, compared to the full image, the patch depicts intensity patterns for a smaller number of different types of bases A, C, T, and G. The patch can depict low-complexity base patterns in which some of the four bases A, C, T, and G are represented at a frequency of less than 15%, 10%, or 5% of all the nucleotides. Low nucleotide diversity in the patches creates intensity patterns that lack signal diversity (contrast), i.e., undiverse intensity patterns.

### Intensity Contextualization Unit (Patch Processing Unit)

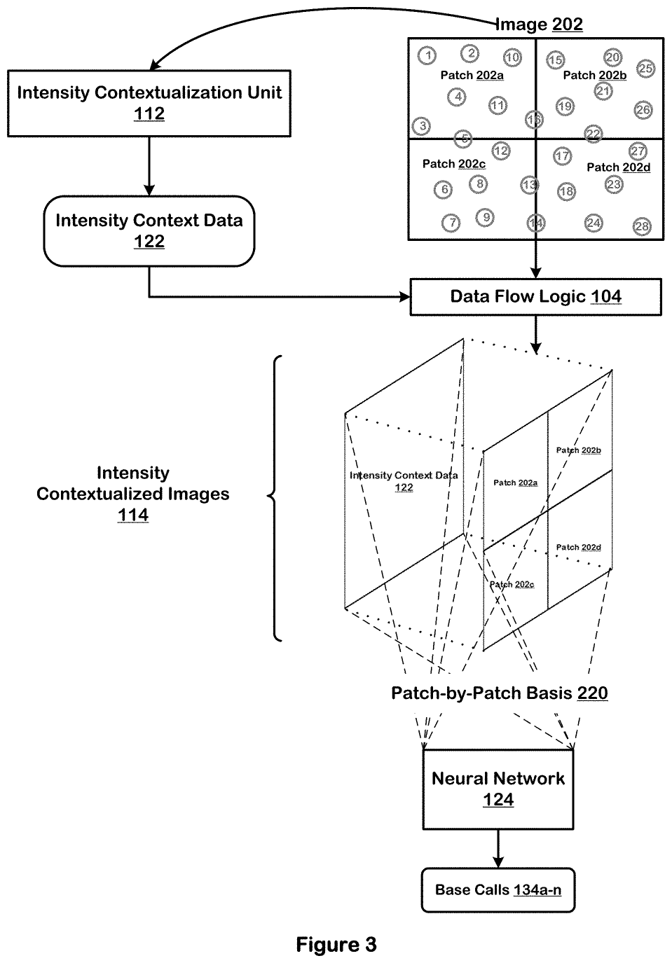

FIG. 3 shows one implementation of generating the intensity contextualized images 114. To compensate for the lack of intensity diversity in the patches, the intensity contextualization unit 112 generates the intensity context data 122 from the images 102 and makes the intensity context data 122 available for incorporation into the patches.

The intensity contextualization unit 112 is configured with feature extraction logic that is applied on the intensity values in the images 102 to generate the intensity context data 122. The feature extraction logic determines summary statistics of the intensity values in the images 102. Examples of the summary statistics include maximum value, minimum value, mean, mode, standard deviation, variance, skewness, kurtosis, percentiles, and entropy. In other implementations, the feature extraction logic determines secondary statistics based on the summary statistics. Examples of the secondary statistics include deltas, sums, series of maximum values, series of minimum values, minimum of the maximum values in the series, and maximum of minimum values in the series.

The intensity context data 122 specifies summary statistics of the intensity values. In one implementation, the intensity context data 122 identifies a maximum value in the intensity values. In one implementation, the intensity context data 122 identifies a minimum value in the intensity values. In one implementation, the intensity context data 122 identifies a mean of the intensity values. In one implementation, the intensity context data 122 identifies a mode of the intensity values. In one implementation, the intensity context data 122 identifies a standard deviation of the intensity values. In one implementation, the intensity context data 122 identifies a variance of the intensity values. In one implementation, the intensity context data 122 identifies a skewness of the intensity values. In one implementation, the intensity context data 122 identifies a kurtosis of the intensity values. In one implementation, the intensity context data 122 identifies an entropy of the intensity values.

In one implementation, the intensity context data 122 identifies one or more percentiles of the intensity values. In one implementation, the intensity context data 122 identifies a delta between at least one of the maximum value and the minimum value, the maximum value and the mean, the mean and the minimum value, and a higher one of the percentiles and a lower one of the percentiles. In one implementation, the intensity context data 122 identifies a sum of the intensity values. In one implementation, the intensity contextualization unit 112 determines a plurality (or series) of maximum values by dividing the intensity values into groups and determining a maximum value for each of the groups. The intensity context data 122 identifies the smallest value in the plurality of maximum values.

In one implementation, the intensity contextualization unit 112 determines a plurality (or series) of minimum values by dividing the intensity values into groups and determining a minimum value for each of the groups. The intensity context data 122 identifies the largest value in the plurality of minimum values. In one implementation, the intensity contextualization unit 112 determines a plurality of sums by dividing the intensity values into groups and determining a sum of intensity values in each of the groups. The intensity context data 122 identifies the smallest value in the plurality of sums. In other implementations, the intensity context data 122 identifies the largest value in the plurality of sums. In yet other implementations, the intensity context data 122 identifies a mean of the plurality of sums.

The intensity context data 122 comprises numerical values (e.g., floating-point numbers or integers) determined (or calculated) from the intensity values in the images 102. In one implementation, the numerical values in the intensity context data 122 are features or feature maps generated as a result of applying convolution operations on the images 102. The features in the intensity context data 122 can be stored as pixelated units (e.g., pixels, superpixels, subpixels) that contain the respective numerical values.

In one implementation, the intensity contextualization unit 112 is a multilayer perceptron (MLP). In another implementation, the intensity contextualization unit 112 is a feedforward neural network. In yet another implementation, the intensity contextualization unit 112 is a fully-connected neural network. In a further implementation, the intensity contextualization unit 112 is a fully convolutional neural network. In yet further implementation, the intensity contextualization unit 112 is a semantic segmentation neural network. In yet another further implementation, the intensity contextualization unit 112 is a generative adversarial network (GAN).

In one implementation, the intensity contextualization unit 112 is a convolutional neural network (CNN) with a plurality of convolution layers. In another implementation, it is a recurrent neural network (RNN) such as a long short-term memory network (LSTM), bi-directional LSTM (Bi-LSTM), or a gated recurrent unit (GRU). In yet another implementation, it includes both a CNN and a RNN.

In yet other implementations, the intensity contextualization unit 112 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. It can use one or more loss functions such as logistic regression/log loss, multi-class cross-entropy/softmax loss, binary cross-entropy loss, mean-squared error loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). It can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

The intensity contextualization unit 112 is trained using backpropagation-based gradient update techniques. Example gradient descent techniques that can be used for training the intensity contextualization unit 112 include stochastic gradient descent, batch gradient descent, and mini-batch gradient descent. Some examples of gradient descent optimization algorithms that can be used to train the intensity contextualization unit 112 are Momentum, Nesterov accelerated gradient, Adagrad, Adadelta, RMSprop, Adam, AdaMax, Nadam, and AMSGrad.

In one implementation, an initial version of intensity context data, as generated by the intensity contextualization unit 112, has spatial dimensions that are different from the images 102 (e.g., the full image 202). In such a case, the initial version of intensity context data produced by the intensity contextualization unit 122 is subjected to further processing to generate the intensity context data 122 that is appendable to the full image 202. In one implementation, the intensity context data 122 “being appendable” to the full image 202 means that the two have matching or similar spatial dimensions, i.e., width and height. The initial version of intensity context data can be converted into the appendable intensity context data 122 by use of dimensionality augmentation techniques like upsampling, deconvolution, transpose convolution, dilated convolution, concatenation, and padding (e.g., when the spatial dimensions of the two are not exactly matching).

For example, the initial version of intensity context data can be of size 1×1, 3×3, or 5×5, whereas the full image 202 is of size 115×115. In this scenario, the initial version of intensity context data is duplicated (or cloned) such that the clones of the initial version of intensity context data are concatenated to form the intensity context data 122, which has spatial dimensions that match the full image 202. Consider, for example, that the spatial dimensions of the initial version of intensity context data are 1×1, and the full image 202 is of size 115×115. Then, additional one hundred and fourteen clones of the initial version of intensity context data are generated and concatenated with each other and with the 1×1 initial version of intensity context data to from a 115×115 grid that has spatial dimensions which match the 115×115 full image 202. This 115×115 grid constitutes the intensity context data 122.

In some implementations, the intensity context data 122 comprises a plurality of context channels. Each context channel in the plurality of context channels is constructed using a respective feature from a plurality of features generated by the intensity contextualization unit 112. Consider, for example, that the intensity contextualization unit 112 generates six 1×1 initial versions of the intensity context data. Then, six 115×115 context channels are generated using concatenation to constitute the intensity context data 122.

The data flow logic 104 appends the intensity context data 122 to the images 102 to generate the intensity contextualized images 114. In one implementation, the intensity context data 122 comprises the plurality of context channels in which each context channel has the same spatial dimensions as the images 202. Consider a full image that has two image channels that form a first grid (or array) of pixelated units of size 115×115 and depth two. Further consider that the intensity context data 122 has six context channels that form a second grid of pixelated units of size 115×115 and depth six. Then, the first and second grids of pixelated units are appended (or attached) on a pixelated unit-by-pixelated unit basis to form a single grid of pixelated units of size 115×115 and depth eight, referred to herein as intensity contextualized images 114. This way, each of the intensity contextualized images has eight channels, two image channels from the full image and six context channels from the intensity context data 122.

The data flow logic 104 provides the intensity contextualized images 114 to the neural network 124 as input, which accesses them on the patch-by-patch basis 220. The input to the neural network 124 comprises intensity contextualized images for multiple sequencing cycles (e.g., a current sequencing cycle, one or more preceding sequencing cycles, and one or more successive sequencing cycles). In one implementation, the input to the neural network 124 comprises intensity contextualized images for three sequencing cycles, such that intensity contextualized images for a current (time t) sequencing cycle to be base called is accompanied with (i) intensity contextualized images for a left flanking/context/previous/preceding/prior (time t−1) sequencing cycle and (ii) intensity contextualized images for a right flanking/context/next/successive/subsequent (time t+1) sequencing cycle. In another implementation, the input to the neural network 124 comprises intensity contextualized images for five sequencing cycles, such that intensity contextualized images for a current (time t) sequencing cycle to be base called is accompanied with (i) data for a first left flanking/context/previous/preceding/prior (time t−1) sequencing cycle, (ii) intensity contextualized images for a second left flanking/context/previous/preceding/prior (time t−2) sequencing cycle, (iii) intensity contextualized images for a first right flanking/context/next/successive/subsequent (time t+1), and (iv) intensity contextualized images for a second right flanking/context/next/successive/subsequent (time t+2) sequencing cycle. In yet another implementation, the input to the neural network 124 comprises intensity contextualized images for seven sequencing cycles, such that data for a current (time t) sequencing cycle to be base called is accompanied with (i) intensity contextualized images for a first left flanking/context/previous/preceding/prior (time t−1) sequencing cycle, (ii) intensity contextualized images for a second left flanking/context/previous/preceding/prior (time t−2) sequencing cycle, (iii) intensity contextualized images for a third left flanking/context/previous/preceding/prior (time t−3) sequencing cycle, (iv) intensity contextualized images for a first right flanking/context/next/successive/subsequent (time t+1), (v) intensity contextualized images for a second right flanking/context/next/successive/subsequent (time t+2) sequencing cycle, and (vi) intensity contextualized images for a third right flanking/context/next/successive/subsequent (time t+3) sequencing cycle. In other implementations, the input to the neural network 124 comprises intensity contextualized images for a single sequencing cycle. In yet other implementations, the input to the neural network 124 comprises intensity contextualized images for 58, 75, 92, 130, 168, 175, 209, 225, 230, 275, 318, 325, 330, 525, or 625 sequencing cycles.

In another implementation, the sequencing images from the current (time t) sequencing cycle are accompanied with the sequencing images from the preceding (time t−1) sequencing cycle and the sequencing images from the succeeding (time t+1) sequencing cycle. The neural network-based base caller 104 processes the sequencing images through its convolution layers and produces an alternative representation, according to one implementation. The alternative representation is then used by an output layer (e.g., a softmax layer) for generating a base call for either just the current (time t) sequencing cycle or each of the sequencing cycles, i.e., the current (time t) sequencing cycle, the preceding (time t−1) sequencing cycle, and the succeeding (time t+1) sequencing cycle. The resulting base calls form the sequencing reads.

Neural Network-Based Base Calling

The neural network-based base caller 124 processes the intensity contextualized images 114 through its convolution layers and produces an alternative representation, according to one implementation. The alternative representation is then used by an output layer (e.g., a softmax layer) for generating a base call for either just the current (time t) sequencing cycle or each of the sequencing cycles, i.e., the current (time t) sequencing cycle, the preceding (time t−1) sequencing cycle, and the succeeding (time t+1) sequencing cycle. The resulting base calls form the sequencing reads and are stored as the base calls 134.

The neural network-based base caller 124 accesses the intensity contextualized images 114 on a patch-by-patch basis (or a tile-by-tile basis). Each of the patches is a sub-grid (or sub-array) of pixelated units in the grid of pixelated units that forms the sequencing images. The patches have dimensions q×r of the sub-grid of pixelated units, where q (width) and r (height) are any numbers ranging from 1 and 10000 (e.g., 3×3, 5×5, 7×7, 10×10, 15×15, 25×25, 64×64, 78×78, 115×115). In some implementations, q and r are the same. In other implementations, q and r are different. In some implementations, the patches extracted from a sequencing image are of the same size. In other implementations, the patches are of different sizes. In some implementations, the patches can have overlapping pixelated units (e.g., on the edges).

In one implementation, the neural network-based base caller 124 outputs a base call for a single target cluster for a particular sequencing cycle. In another implementation, it outputs a base call for each target cluster in a plurality of target clusters for the particular sequencing cycle. In yet another implementation, it outputs a base call for each target cluster in a plurality of target clusters for each sequencing cycle in a plurality of sequencing cycles, thereby producing a base call sequence for each target cluster.

In one implementation, the neural network-based base caller 124 is a multilayer perceptron (MLP). In another implementation, the neural network-based base caller 124 is a feedforward neural network. In yet another implementation, the neural network-based base caller 124 is a fully-connected neural network. In a further implementation, the neural network-based base caller 124 is a fully convolutional neural network. In yet further implementation, the neural network-based base caller 124 is a semantic segmentation neural network. In yet another further implementation, the neural network-based base caller 124 is a generative adversarial network (GAN).

In one implementation, the neural network-based base caller 124 is a convolutional neural network (CNN) with a plurality of convolution layers. In another implementation, it is a recurrent neural network (RNN) such as a long short-term memory network (LSTM), bi-directional LSTM (Bi-LSTM), or a gated recurrent unit (GRU). In yet another implementation, it includes both a CNN and a RNN.

In yet other implementations, the neural network-based base caller 124 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. It can use one or more loss functions such as logistic regression/log loss, multi-class cross-entropy/softmax loss, binary cross-entropy loss, mean-squared error loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). It can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

The neural network-based base caller 124 is trained using backpropagation-based gradient update techniques. Example gradient descent techniques that can be used for training the neural network-based base caller 124 include stochastic gradient descent, batch gradient descent, and mini-batch gradient descent. Some examples of gradient descent optimization algorithms that can be used to train the neural network-based base caller 124 are Momentum, Nesterov accelerated gradient, Adagrad, Adadelta, RMSprop, Adam, AdaMax, Nadam, and AMSGrad.

Additional details about the neural network-based base caller 124 can be found in U.S. Provisional Patent Application No. 62/821,766, titled “ARTIFICIAL INTELLIGENCE-BASED SEQUENCING,” (Attorney Docket No. ILLM 1008-9/IP-1752-PRV), filed on Mar. 21, 2019, which is incorporated herein by reference.

In some implementations, the intensity contextualization unit 112 contains intensity extractor, discriminators, and approximators (e.g., convolution filters) whose kernel weights or coefficients can be learned (or trained) using backpropagation-based gradient update techniques. In such implementations, the intensity contextualization unit 112 is trained “end-to-end” with the neural network 124, such that the error is calculated between the base call predictions of the neural network 124 and the ground truth base calls and the gradients determined from the error are used to update the weights of the neural network 124 and further update the weights of the intensity contextualization unit 112. This way, the intensity contextualization unit 112 learns to extract those intensity features and contexts from the images 102 that contribute to correct base call predictions by the neural network 124.

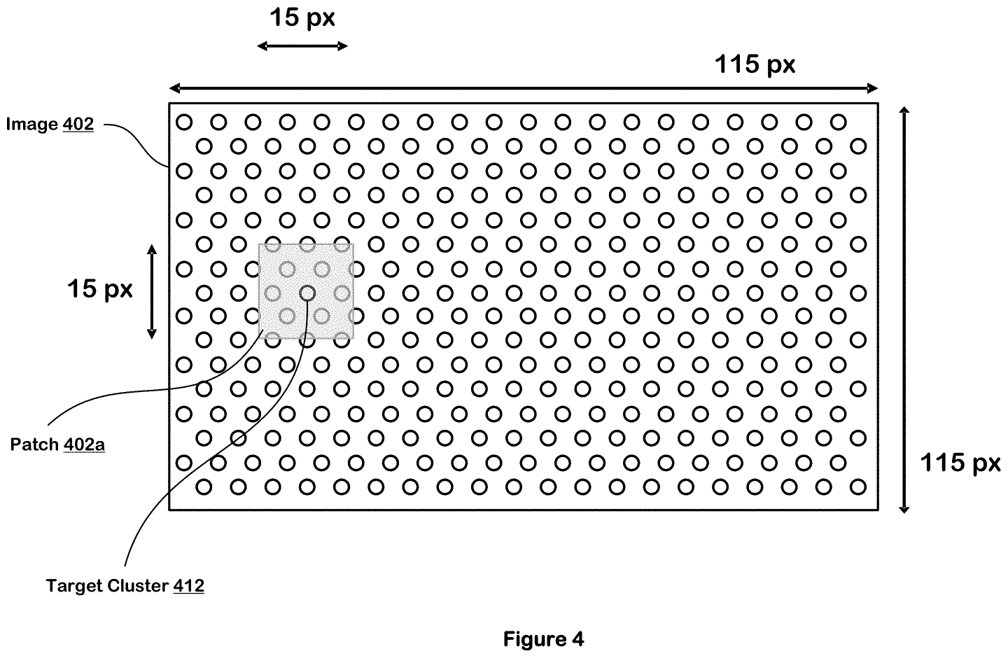

FIG. 4 shows one example of a full image 402 from which a patch 402a is accessed such that the patch 402a is centered at a target cluster 412 (in red) to be base called. The size of the full image 402 is 115×115 pixels and the size of the patch 402a is 15×15 pixels.

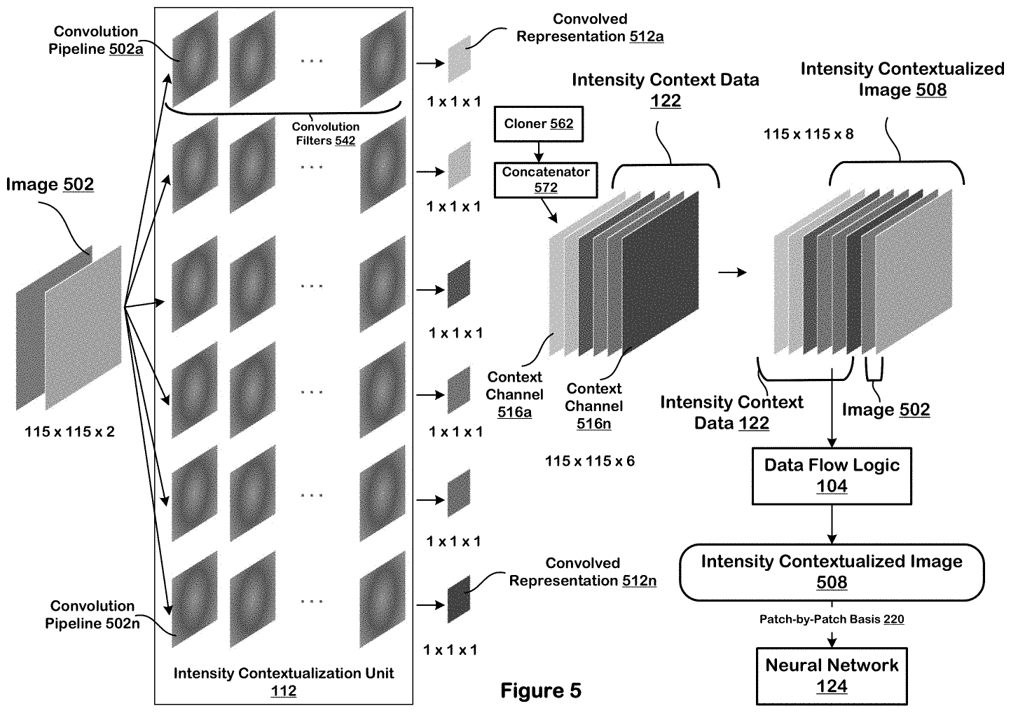

FIG. 5 depicts one implementation of the intensity contextualization unit 112 having a plurality of convolution pipelines. Each of the convolution pipelines has a plurality of convolution filters. Convolution filters in the plurality of convolution filters have varying filter sizes and varying filter strides. Each of the convolution pipelines processes an image to generate a plurality of convolved representations of the image.

In the illustrated example, the input to the intensity contextualization unit 112 is a full image 502 of size 115×115 pixels and two image channels, i.e., blue and green image channels. The intensity contextualization unit 112 has n convolution pipelines 502a, . . . , 502n, where n can range from 1 to 100 (e.g., 4, 6, 10, 16, 18, 20). A convolution pipeline has a series of convolution filters (e.g., 542). In some implementations, convolution filters in the series of convolution filters of a particular convolution pipeline have different filter (or kernel) sizes. In other implementations, convolution filters in the series of convolution filters of a particular convolution pipeline have same filter sizes. In one example, the particular convolution pipeline can have three sets of filters, such that filters in a first set of filters are of size 3×3, filters in a second set of filters are of size 3×3, and filters in a third set of filters are of size 12×12. In another example, the particular convolution pipeline can have three sets of filters, such that filters in a first set of filters are of size 3×3, filters in a second set of filters are of size 4×4, and filters in a third set of filters are of size 9×9. In yet another example, the particular convolution pipeline can have four sets of filters, such that filters in a first set of filters are of size 3×3, filters in a second set of filters are of size 3×3, filters in a third set of filters are of size 4×4, and filters in a fourth set of filters are of size 9×9. In yet another example, the particular convolution pipeline can have four sets of filters, such that filters in a first set of filters are of size 5×5, filters in a second set of filters are of size 3×3, filters in a third set of filters are of size 3×3, and filters in a fourth set of filters are of size 7×7. In yet another example, the particular convolution pipeline can have four sets of filters, such that filters in a first set of filters are of size 5×5, filters in a second set of filters are of size 3×3, filters in a third set of filters are of size 3×3, and filters in a fourth set of filters are of size 7×7. In yet another example, the particular convolution pipeline can have four sets of filters, such that filters in a first set of filters are of size 5×5, filters in a second set of filters are of size 4×4, filters in a third set of filters are of size 4×4, and filters in a fourth set of filters are of size 5×5. In yet another example, the particular convolution pipeline can have four sets of filters, such that filters in a first set of filters are of size 5×5, filters in a second set of filters are of size 5×5, filters in a third set of filters are of size 5×5, and filters in a fourth set of filters are of size 3×3. In yet another example, the particular convolution pipeline can have four sets of filters, such that filters in a first set of filters are of size 3×3, filters in a second set of filters are of size 3×3, filters in a third set of filters are of size 4×4, and filters in a fourth set of filters are of size 9×9.

In some implementations, convolution filters in the series of convolution filters of a particular convolution pipeline have different stride sizes. In one example, the particular convolution pipeline can have three sets of filters, such that filters in a first set of filters use a stride size of 3, filters in a second set of filters use a stride size of 4, and filters in a third set of filters use a stride size of 1. In other implementations, convolution filters in the series of convolution filters of a particular convolution pipeline have same stride sizes.

The image 502 is fed as input to each of the n convolution pipelines 502a, . . . , 502n. Each convolution pipeline processes the image 502, generates successive feature maps, and produces a final output (e.g., convolved representations 512a, 512n of size 1×1). Because the kernel weights or coefficients of the convolution filters vary across the convolution pipelines, respective final outputs of the convolution pipelines are also different and therefore encode different intensity features or contexts determined from the intensity values in the image 502. This way, a plurality of intensity features and contexts is determined from the image 502 by using the plurality of convolution pipelines configured with varying convolution coefficients (or kernel weights). Each of the final outputs is made up of one or more pixelated units (e.g., pixels, superpixels, subpixels).

In some implementations, the spatial dimensions of the respective final outputs (e.g., 512a, 512n) of the n convolution pipelines 502a, . . . , 502n are cloned and concatenated by a cloner 562 and concatenator 572 to match the spatial dimensions of the image 502, as discussed above. Then, the cloned and concatenated versions of the respective final outputs form respective context channels (e.g., 516a, 516n), which are arranged on a pixelated unit-by-pixelated unit basis to constitute the intensity context data 122 of size 115×115×6. Then, the intensity context data 122 is appended to the image 502 on the pixelated unit-by-pixelated unit basis to form intensity contextualized image 508 of size 115×115×8, of which the six channels are the context channels from the intensity context data 122 and the two channels are image channels from the image 502.

The data flow logic 104 provides the intensity contextualized image 508 as input to the neural network 124 for base calling, which accesses and base calls the intensity contextualized image 508 on the patch-by-patch basis 220.

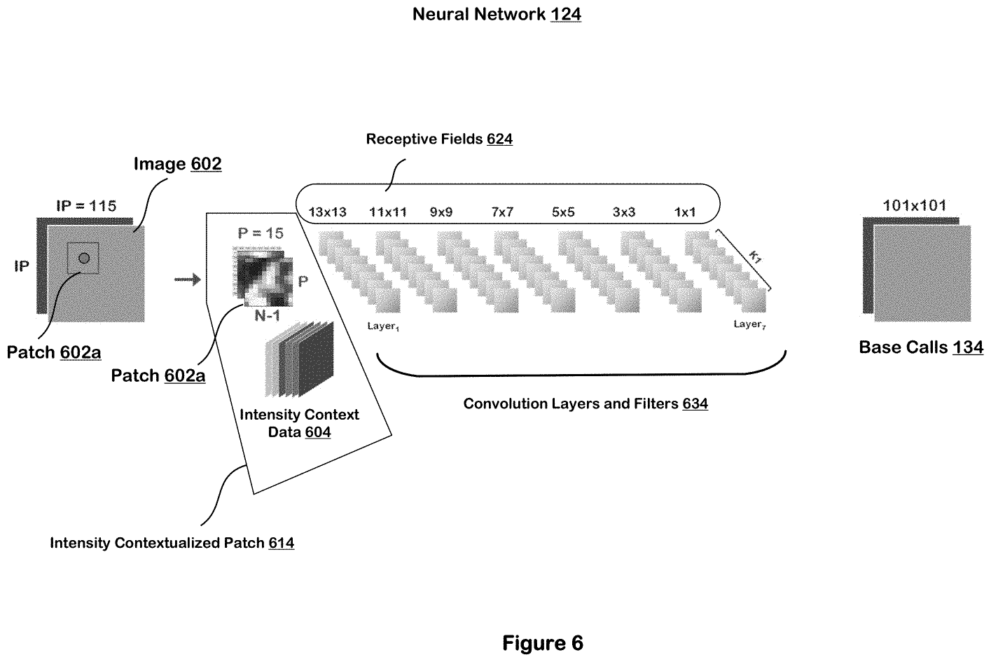

FIG. 6 illustrates one implementation of the neural network 124 processing an intensity contextualized patch 614 and generating the base calls 134. In the illustrated example, a patch 602a of size 15×15 is accessed from a full image 602 of size 115×115. Then, the intensity context data 604 of size 15×15 is pixelwise appended to the patch 602a to form the intensity contextualized patch 614. The neural network 124 comprises a plurality of convolution layers and filters 634 whose receptive fields 624 are smaller than the full image 602. As a result, without the intensity context data 604 determined from the full image 602, when the convolution layers and filters 634 analyze the patch 602a, their receptive fields 624 are confined to the spatial dimensions of the patch 602a and therefore do not take into account image portions of the full image 602 that are outside the patch 602a. To compensate for the confined receptive fields 624, the intensity context data 604 provides intensity context from the distant regions of the image that are not covered by the patch 602a. The intensity contextualized patch 614 is processed by the convolution layers and filters 634 of the neural network 124 to generate the base calls 134.

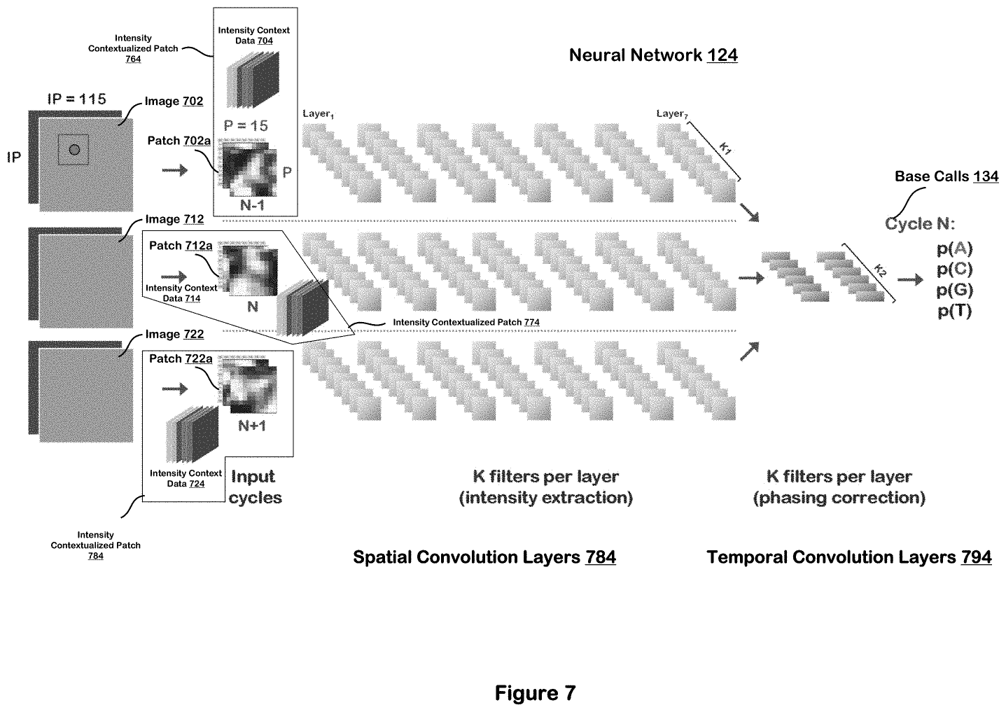

FIG. 7 shows one implementation of the neural network 124 processing previous, current, and successive intensity contextualized images 764, 774, 784 for a plurality of sequencing cycles and generating the base calls 134. Image 702 is generated at a previous sequencing cycle t−1 of a sequencing run. Image 712 is generated at a current sequencing cycle t of the sequencing run. Image 722 is generated at a successive sequencing cycle t+1 of the sequencing run. Previous patch 702a is accessed from the previous image 702 and previous intensity context data 704 is determined from the intensity values in the previous image 702 and pixelwise appended to the previous patch 702a to form the previous intensity contextualized patch 764. Current patch 712a is accessed from the current image 712 and current intensity context data 704 is determined from the intensity values in the current image 712 and pixelwise appended to the current patch 712a to form the current intensity contextualized patch 774. Successive patch 722a is accessed from the successive image 722 and successive intensity context data 714 is determined from the intensity values in the successive image 722 and pixelwise appended to the successive patch 722a to form the successive intensity contextualized patch 784.

The neural network 124 uses a specialized architecture to segregate processing of data for different sequencing cycles. The motivation for using the specialized architecture is described first. As discussed above, the neural network 124 processes intensity contextualized images for a current sequencing cycle, one or more preceding sequencing cycles, and one or more successive sequencing cycles. Data for additional sequencing cycles provides sequence-specific context. The neural network-based base caller 124 learns the sequence-specific context during training and base call them. Furthermore, data for pre and post sequencing cycles provides second order contribution of pre-phasing and phasing signals to the current sequencing cycle.

However, images captured at different sequencing cycles and in different image channels are misaligned and have residual registration error with respect to each other. To account for this misalignment, the specialized architecture comprises spatial convolution layers that do not mix information between sequencing cycles and only mix information within a sequencing cycle.

Spatial convolution layers use so-called “segregated convolutions” that operationalize the segregation by independently processing data for each of a plurality of sequencing cycles through a “dedicated, non-shared” sequence of convolutions. The segregated convolutions convolve over data and resulting feature maps of only a given sequencing cycle, i.e., intra-cycle, without convolving over data and resulting feature maps of any other sequencing cycle.

Consider, for example, that the input data comprises (i) current intensity contextualized patch for a current (time t) sequencing cycle to be base called, (ii) previous intensity contextualized patch for a previous (time t−1) sequencing cycle, and (iii) next intensity contextualized patch for a next (time t+1) sequencing cycle. The specialized architecture then initiates three separate convolution pipelines, namely, a current convolution pipeline, a previous convolution pipeline, and a next convolution pipeline. The current data processing pipeline receives as input the current intensity contextualized patch for the current (time t) sequencing cycle and independently processes it through a plurality of spatial convolution layers 784 to produce a so-called “current spatially convolved representation” as the output of a final spatial convolution layer. The previous convolution pipeline receives as input the previous intensity contextualized patch for the previous (time t−1) sequencing cycle and independently processes it through the plurality of spatial convolution layers 784 to produce a so-called “previous spatially convolved representation” as the output of the final spatial convolution layer. The next convolution pipeline receives as input the next intensity contextualized patch for the next (time t+1) sequencing cycle and independently processes it through the plurality of spatial convolution layers 784 to produce a so-called “next spatially convolved representation” as the output of the final spatial convolution layer.

In some implementations, the current, previous, and next convolution pipelines are executed in parallel. In some implementations, the spatial convolution layers are part of a spatial convolutional network (or subnetwork) within the specialized architecture.

The neural network-based base caller 124 further comprises temporal convolution layers 794 that mix information between sequencing cycles, i.e., inter-cycles. The temporal convolution layers 794 receive their inputs from the spatial convolutional network and operate on the spatially convolved representations produced by the final spatial convolution layer for the respective data processing pipelines.

The inter-cycle operability freedom of the temporal convolution layers emanates from the fact that the misalignment property, which exists in the image data fed as input to the spatial convolutional network, is purged out from the spatially convolved representations by the stack, or cascade, of segregated convolutions performed by the sequence of spatial convolution layers.

Temporal convolution layers 794 use so-called “combinatory convolutions” that groupwise convolve over input channels in successive inputs on a sliding window basis. In one implementation, the successive inputs are successive outputs produced by a previous spatial convolution layer or a previous temporal convolution layer.

In some implementations, the temporal convolution layers 794 are part of a temporal convolutional network (or subnetwork) within the specialized architecture. The temporal convolutional network receives its inputs from the spatial convolutional network. In one implementation, a first temporal convolution layer of the temporal convolutional network groupwise combines the spatially convolved representations between the sequencing cycles. In another implementation, subsequent temporal convolution layers of the temporal convolutional network combine successive outputs of previous temporal convolution layers. The output of the final temporal convolution layer is fed to an output layer that produces an output. The output is used to base call one or more clusters at one or more sequencing cycles.

### Performance Results as Objective Indicia of Inventiveness and Non-Obviousness

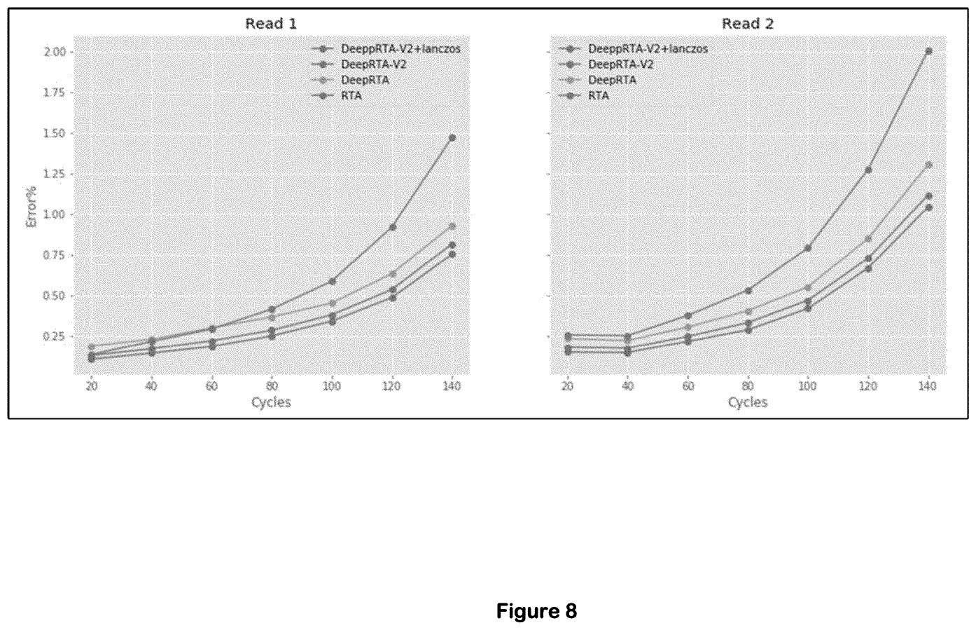

FIG. 8 compares the base calling accuracy of the disclosed neural network-based base caller configured with the disclosed intensity contextualization unit (referred to herein as “DeepRTA-V2”) against a neural network-based base caller without the disclosed intensity contextualization unit (referred to herein as “DeepRTA”). Additional details about DeepRTA can be found in commonly owned U.S. patent application Ser. Nos. 16/825,987; 16/825,991; 16/826,126; 16/826,134; 16/826,168; 62/979,412; 62/979,411; 17/179,395; 62/979,399; 17/180,480; 17/180,513; 62/979,414; 62/979,385; and 63/072,032.

FIG. 8 also compares the base calling accuracy of DeepRTA-V2 against a non-neural network-based base caller without the disclosed intensity contextualization unit (referred to herein as “RTA”). Additional details about RTA can be found in commonly owned U.S. patent application Ser. No. 13/006,206. In FIG. 8, the model titled “DeepRTA-V2+lanczos” is the disclosed neural network-based base caller with the disclosed intensity contextualization unit combined with an additional non-linearity logic referred to herein as “lanczos.”

Therefore, in FIG. 8, we provide base calling performance comparison of DeepRTA-V2 against another neural-network based base caller (DeepRTA) and a non-neural network-based base caller (RTA). As demonstrated by FIG. 8, superior base calling performance of DeepRTA-V2 against these benchmark models is an objective indication of the inventive and non-obvious character of the disclosed intensity contextualization unit.

In FIG. 8, the y-axis has the base calling error rate (“Error %”). The Error % is calculated over a multitude of base calls made for a multitude of clusters (e.g., hundreds of or millions of base calls made for hundreds of or millions of clusters). Also, in FIG. 8, the x-axis has the progression of sequencing cycles 20-140 of a sequencing run over which the multitude of base calls were made for reads 1 and 2.

In FIG. 8, the Error % of RTA is depicted by grey fitted lines; the Error % of DeepRTA is depicted by purple fitted lines; the Error % of DeepRTA-V2 is depicted by cyan fitted lines; and the Error % of DeepRTA-V2+lanczos is depicted by red fitted lines. As demonstrated in FIG. 8, DeepRTA-V2 has lower base calling error rates than DeepRTA and RTA. Furthermore, this is true consistently for the progression of the sequencing cycles 20-140 for both reads 1 and 2, as indicated by the cyan and red fitted lines being consistently below the grey and purple fitted lines in FIG. 8.

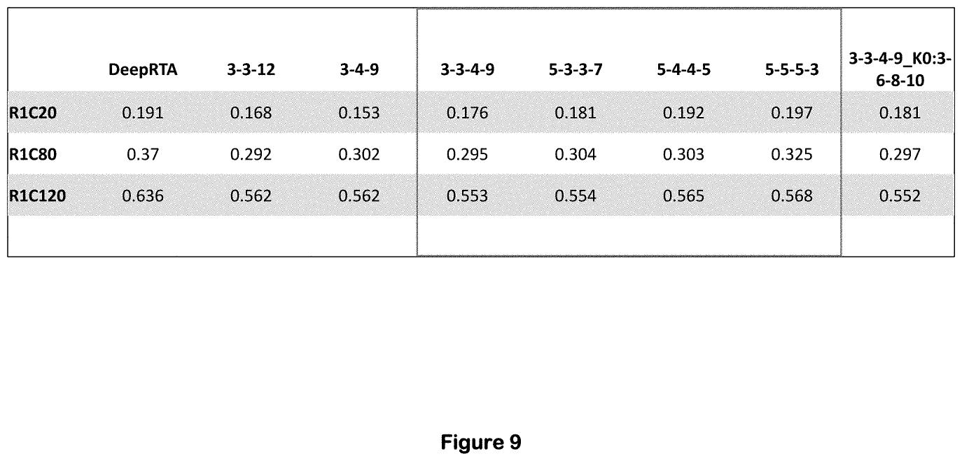

FIG. 9 shows the base calling error rates observed for various combinations (configurations) of filter sizes (or kernel sizes), strides, and filter bank sizes (K) of convolution filters of the disclosed neural network-based base caller 124.

In FIG. 9, “R1C20” denotes sequencing cycle twenty during sequencing of read 1. In particular, R1C20 denotes a multitude of base calls made during the sequencing cycle twenty for a multitude of clusters (e.g., hundreds of or millions of base calls made for hundreds of or millions of clusters). In FIG. 9, R1C20 is used as a representative sequence cycle for early sequencing cycles in a sequencing run.

In FIG. 9, “R1C80” denotes sequencing cycle eighty during the sequencing of read 1. In particular, R1C80 denotes a multitude of base calls made during the sequencing cycle eighty for the multitude of clusters (e.g., hundreds of or millions of base calls made for hundreds of or millions of clusters). In FIG. 9, R1C80 is used as a representative sequence cycle for middle sequencing cycles in the sequencing run.

In FIG. 9, “R1C120” denotes sequencing cycle one hundred and twenty during the sequencing of read 1. In particular, R1C120 denotes a multitude of base calls made during the sequencing cycle one hundred and twenty for the multitude of clusters (e.g., hundreds of or millions of base calls made for hundreds of or millions of clusters). In FIG. 9, R1C120 is used as a representative sequence cycle for later sequencing cycles in the sequencing run.

In FIG. 9, “DeepRTA” denotes a particular combination of filter sizes (or kernel sizes), strides, and filter bank sizes (K) of convolution filters DeepRTA.

In FIG. 9, the “3-3-12” combination denotes three successive spatial convolution layers of the disclosed neural network-based base caller 124. The three successive spatial convolution layers are arranged in a sequence, such that a patch is first processed by a first spatial convolution layer to produce a first intermediate output, then the first intermediate output is processed by a second convolution layer to produce a second intermediate output, and then the second intermediate output is processed by a third spatial convolution layer to produce a third intermediate output. The first spatial convolution layer has convolution filters/kernels of size 3×3. The second spatial convolution layer has convolution filters/kernels of size 3×3. The third spatial convolution layer has convolution filters/kernels of size 12×12. In some implementations, the first, second, and third spatial convolution layers can use different striding so that the third intermediate output has a target dimensionality (e.g., 1×1 or 2×2). In other implementations, the first, second, and third spatial convolution layers can use a same striding so that the third intermediate output has the target dimensionality (e.g., 1×1 or 2×2). In some implementations, the first, second, and third spatial convolution layers can use different padding so that the third intermediate output has the target dimensionality (e.g., 1×1 or 2×2). In other implementations, the first, second, and third spatial convolution layers can use a same padding so that the third intermediate output has the target dimensionality (e.g., 1×1 or 2×2). In some implementations, the first, second, and third spatial convolution layers can use different filter bank sizes so that the third intermediate output has the target dimensionality (e.g., 1×1 or 2×2). In other implementations, the first, second, and third spatial convolution layers can use a same filter bank size (e.g., six or ten) so that the third intermediate output has the target dimensionality (e.g., 1×1 or 2×2).

In FIG. 9, the “3-4-9” combination denotes three successive spatial convolution layers of the disclosed neural network-based base caller 124. The three successive spatial convolution layers are arranged in a sequence, such that a patch is first processed by a first spatial convolution layer to produce a first intermediate output, then the first intermediate output is processed by a second convolution layer to produce a second intermediate output, and then the second intermediate output is processed by a third spatial convolution layer to produce a third intermediate output. The first spatial convolution layer has convolution filters/kernels of size 3×3. The second spatial convolution layer has convolution filters/kernels of size 4×4. The third spatial convolution layer has convolution filters/kernels of size 9×9. In some implementations, the first, second, and third spatial convolution layers can use different striding so that the third intermediate output has a target dimensionality (e.g., 1×1 or 2×2). In other implementations, the first, second, and third spatial convolution layers can use a same striding so that the third intermediate output has the target dimensionality (e.g., 1×1 or 2×2). In some implementations, the first, second, and third spatial convolution layers can use different padding so that the third intermediate output has the target dimensionality (e.g., 1×1 or 2×2). In other implementations, the first, second, and third spatial convolution layers can use a same padding so that the third intermediate output has the target dimensionality (e.g., 1×1 or 2×2). In some implementations, the first, second, and third spatial convolution layers can use different filter bank sizes so that the third intermediate output has the target dimensionality (e.g., 1×1 or 2×2). In other implementations, the first, second, and third spatial convolution layers can use a same filter bank size (e.g., six or ten) so that the third intermediate output has the target dimensionality (e.g., 1×1 or 2×2).

In FIG. 9, the “3-3-4-9” combination denotes four successive spatial convolution layers of the disclosed neural network-based base caller 124. The four successive spatial convolution layers are arranged in a sequence, such that a patch is first processed by a first spatial convolution layer to produce a first intermediate output, then the first intermediate output is processed by a second convolution layer to produce a second intermediate output, then the second intermediate output is processed by a third spatial convolution layer to produce a third intermediate output, and then the third intermediate output is processed by a fourth spatial convolution layer to produce a fourth intermediate output. The first spatial convolution layer has convolution filters/kernels of size 3×3. The second spatial convolution layer has convolution filters/kernels of size 3×3. The third spatial convolution layer has convolution filters/kernels of size 4×4. The fourth spatial convolution layer has convolution filters/kernels of size 9×9. In some implementations, the first, second, third, and fourth spatial convolution layers can use different striding so that the third intermediate output has a target dimensionality (e.g., 1×1 or 2×2). In other implementations, the first, second, third, and fourth spatial convolution layers can use a same striding so that the third intermediate output has the target dimensionality (e.g., 1×1 or 2×2). In some implementations, the first, second, third, and fourth spatial convolution layers can use different padding so that the third intermediate output has the target dimensionality (e.g., 1×1 or 2×2). In other implementations, the first, second, third, and fourth spatial convolution layers can use a same padding so that the third intermediate output has the target dimensionality (e.g., 1×1 or 2×2). In some implementations, the first, second, third, and fourth spatial convolution layers can use different filter bank sizes so that the third intermediate output has the target dimensionality (e.g., 1×1 or 2×2). In other implementations, the first, second, third, and fourth spatial convolution layers can use a same filter bank size (e.g., six or ten) so that the third intermediate output has the target dimensionality (e.g., 1×1 or 2×2).

In FIG. 9, the “5-3-3-7” combination denotes four successive spatial convolution layers of the disclosed neural network-based base caller 124. The four successive spatial convolution layers are arranged in a sequence, such that a patch is first processed by a first spatial convolution layer to produce a first intermediate output, then the first intermediate output is processed by a second convolution layer to produce a second intermediate output, then the second intermediate output is processed by a third spatial convolution layer to produce a third intermediate output, and then the third intermediate output is processed by a fourth spatial convolution layer to produce a fourth intermediate output. The first spatial convolution layer has convolution filters/kernels of size 5×5. The second spatial convolution layer has convolution filters/kernels of size 3×3. The third spatial convolution layer has convolution filters/kernels of size 3×3. The fourth spatial convolution layer has convolution filters/kernels of size 7×7. In some implementations, the first, second, third, and fourth spatial convolution layers can use different striding so that the third intermediate output has a target dimensionality (e.g., 1×1 or 2×2). In other implementations, the first, second, third, and fourth spatial convolution layers can use a same striding so that the third intermediate output has the target dimensionality (e.g., 1×1 or 2×2). In some implementations, the first, second, third, and fourth spatial convolution layers can use different padding so that the third intermediate output has the target dimensionality (e.g., 1×1 or 2×2). In other implementations, the first, second, third, and fourth spatial convolution layers can use a same padding so that the third intermediate output has the target dimensionality (e.g., 1×1 or 2×2). In some implementations, the first, second, third, and fourth spatial convolution layers can use different filter bank sizes so that the third intermediate output has the target dimensionality (e.g., 1×1 or 2×2). In other implementations, the first, second, third, and fourth spatial convolution layers can use a same filter bank size (e.g., six or ten) so that the third intermediate output has the target dimensionality (e.g., 1×1 or 2×2).

In FIG. 9, the “5-4-4-5” combination denotes four successive spatial convolution layers of the disclosed neural network-based base caller 124. The four successive spatial convolution layers are arranged in a sequence, such that a patch is first processed by a first spatial convolution layer to produce a first intermediate output, then the first intermediate output is processed by a second convolution layer to produce a second intermediate output, then the second intermediate output is processed by a third spatial convolution layer to produce a third intermediate output, and then the third intermediate output is processed by a fourth spatial convolution layer to produce a fourth intermediate output. The first spatial convolution layer has convolution filters/kernels of size 5×5. The second spatial convolution layer has convolution filters/kernels of size 4×4. The third spatial convolution layer has convolution filters/kernels of size 4×4. The fourth spatial convolution layer has convolution filters/kernels of size 5×5. In some implementations, the first, second, third, and fourth spatial convolution layers can use different striding so that the third intermediate output has a target dimensionality (e.g., 1×1 or 2×2). In other implementations, the first, second, third, and fourth spatial convolution layers can use a same striding so that the third intermediate output has the target dimensionality (e.g., 1×1 or 2×2). In some implementations, the first, second, third, and fourth spatial convolution layers can use different padding so that the third intermediate output has the target dimensionality (e.g., 1×1 or 2×2). In other implementations, the first, second, third, and fourth spatial convolution layers can use a same padding so that the third intermediate output has the target dimensionality (e.g., 1×1 or 2×2). In some implementations, the first, second, third, and fourth spatial convolution layers can use different filter bank sizes so that the third intermediate output has the target dimensionality (e.g., 1×1 or 2×2). In other implementations, the first, second, third, and fourth spatial convolution layers can use a same filter bank size (e.g., six or ten) so that the third intermediate output has the target dimensionality (e.g., 1×1 or 2×2).

In FIG. 9, the “5-5-5-3” combination denotes four successive spatial convolution layers of the disclosed neural network-based base caller 124. The four successive spatial convolution layers are arranged in a sequence, such that a patch is first processed by a first spatial convolution layer to produce a first intermediate output, then the first intermediate output is processed by a second convolution layer to produce a second intermediate output, then the second intermediate output is processed by a third spatial convolution layer to produce a third intermediate output, and then the third intermediate output is processed by a fourth spatial convolution layer to produce a fourth intermediate output. The first spatial convolution layer has convolution filters/kernels of size 5×5. The second spatial convolution layer has convolution filters/kernels of size 5×5. The third spatial convolution layer has convolution filters/kernels of size 5×5. The fourth spatial convolution layer has convolution filters/kernels of size 3×3. In some implementations, the first, second, third, and fourth spatial convolution layers can use different striding so that the third intermediate output has a target dimensionality (e.g., 1×1 or 2×2). In other implementations, the first, second, third, and fourth spatial convolution layers can use a same striding so that the third intermediate output has the target dimensionality (e.g., 1×1 or 2×2). In some implementations, the first, second, third, and fourth spatial convolution layers can use different padding so that the third intermediate output has the target dimensionality (e.g., 1×1 or 2×2). In other implementations, the first, second, third, and fourth spatial convolution layers can use a same padding so that the third intermediate output has the target dimensionality (e.g., 1×1 or 2×2). In some implementations, the first, second, third, and fourth spatial convolution layers can use different filter bank sizes so that the third intermediate output has the target dimensionality (e.g., 1×1 or 2×2). In other implementations, the first, second, third, and fourth spatial convolution layers can use a same filter bank size (e.g., six or ten) so that the third intermediate output has the target dimensionality (e.g., 1×1 or 2×2).

In FIG. 9, the “3-3-4-9_K0:3-6-8-10” combination denotes four successive spatial convolution layers of the disclosed neural network-based base caller 124. The four successive spatial convolution layers are arranged in a sequence, such that a patch is first processed by a first spatial convolution layer to produce a first intermediate output, then the first intermediate output is processed by a second convolution layer to produce a second intermediate output, then the second intermediate output is processed by a third spatial convolution layer to produce a third intermediate output, and then the third intermediate output is processed by a fourth spatial convolution layer to produce a fourth intermediate output. The first spatial convolution layer has convolution filters/kernels of size 3×3. The second spatial convolution layer has convolution filters/kernels of size 3×3. The third spatial convolution layer has convolution filters/kernels of size 4×4. The fourth spatial convolution layer has convolution filters/kernels of size 9×9. In some implementations, the first, second, third, and fourth spatial convolution layers can use different striding so that the third intermediate output has a target dimensionality (e.g., 1×1 or 2×2). In other implementations, the first, second, third, and fourth spatial convolution layers can use a same striding so that the third intermediate output has the target dimensionality (e.g., 1×1 or 2×2). In some implementations, the first, second, third, and fourth spatial convolution layers can use different padding so that the third intermediate output has the target dimensionality (e.g., 1×1 or 2×2). In other implementations, the first, second, third, and fourth spatial convolution layers can use a same padding so that the third intermediate output has the target dimensionality (e.g., 1×1 or 2×2). The “3-3-4-9_K0:3-6-8-10” combination has a filter bank size of three (i.e., K0=3) in the first spatial convolution layer. The “3-3-4-9_K0:3-6-8-10” combination has a filter bank size of six (i.e., K0=6) in the second spatial convolution layer. The “3-3-4-9_K0:3-6-8-10” combination has a filter bank size of eight (i.e., K0=8) in the third spatial convolution layer. The “3-3-4-9_K0:3-6-8-10” combination has a filter bank size of ten (i.e., K0=10) in the fourth spatial convolution layer.

In FIG. 9, the values in the table are the respective base calling error rates for the respective combinations (configurations). As demonstrated, the base calling error rates of many of the combinations of the disclosed neural network-based base caller 124 are lower than the DeepRTA. Also, within the different combinations (configurations) of the disclosed neural network-based base caller 124, base calling error rates decrease when the filter/kernel size progressively increases between successive spatial convolution layers.

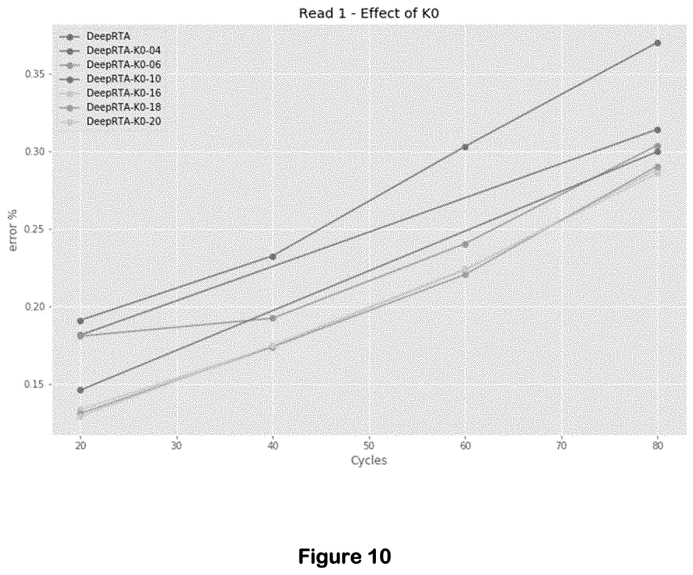

FIG. 10 compares base calling error rate of DeepRTA against base calling error rates of different filter bank size configurations (K0s) of the disclosed neural network-based base caller configured with the disclosed intensity contextualization unit (DeepRTA-K0-04; DeepRTA-K0-06; DeepRTA-K0-10; DeepRTA-K0-16; DeepRTA-K0-18; and DeepRTA-K0-20).

DeepRTA-K0-04 denotes the disclosed neural network-based base caller configured with the disclosed intensity contextualization unit having four convolution filters in each of its n spatial convolution layers (i.e., a filter bank size of four/K0=4). DeepRTA-K0-06 denotes the disclosed neural network-based base caller configured with the disclosed intensity contextualization unit having six convolution filters in each of its n spatial convolution layers (i.e., a filter bank size of six/K0=6). DeepRTA-K0-10 denotes the disclosed neural network-based base caller configured with the disclosed intensity contextualization unit having ten convolution filters in each of its n spatial convolution layers (i.e., a filter bank size of ten/K0=10). DeepRTA-K0-16 denotes the disclosed neural network-based base caller configured with the disclosed intensity contextualization unit having sixteen convolution filters in each of its n spatial convolution layers (i.e., a filter bank size of sixteen/K0=16). DeepRTA-K0-18 denotes the disclosed neural network-based base caller configured with the disclosed intensity contextualization unit having eighteen convolution filters in each of its n spatial convolution layers (i.e., a filter bank size of eighteen/K0=18). DeepRTA-K0-18 denotes the disclosed neural network-based base caller configured with the disclosed intensity contextualization unit having twenty convolution filters in each of its n spatial convolution layers (i.e., a filter bank size of twenty/K0=20).

In FIG. 10, the y-axis has the base calling error rate (“Error %”). The Error % is calculated over a multitude of base calls made for a multitude of clusters (e.g., hundreds of or millions of base calls made for hundreds of or millions of clusters). Also, in FIG. 10, the x-axis has the progression of sequencing cycles 20-80 of a sequencing run over which the multitude of base calls were made for read 1.

In FIG. 10, the Error % of DeepRTA is depicted by a red fitted line; the Error % of DeepRTA-K0-04 is depicted by a blue fitted line; the Error % of DeepRTA-K0-06 is depicted by a purple fitted line; the Error % of DeepRTA-K0-10 is depicted by a grey fitted line; the Error % of DeepRTA-K0-16 is depicted by an orange fitted line; the Error % of DeepRTA-K0-18 is depicted by a green fitted line; and the Error % of DeepRTA-K0-20 is depicted by a pink fitted line.

As demonstrated in FIG. 10, the different filter bank size configurations of the disclosed neural network-based base caller configured with the disclosed intensity contextualization unit, i.e., DeepRTA-K0-04, DeepRTA-K0-06, DeepRTA-K0-10, DeepRTA-K0-16, DeepRTA-K0-18, and DeepRTA-K0-20 have lower base calling error rates than DeepRTA. Furthermore, this is true consistently for the progression of the sequencing cycles 20-80 for read 1, as indicated by the blue, purple, grey, orange, green, and pink fitted lines being consistently below the red fitted line in FIG. 10.

As demonstrated by FIG. 10, superior base calling performance of the different filter bank size configurations of the disclosed neural network-based base caller configured with the disclosed intensity contextualization unit, i.e., DeepRTA-K0-04, DeepRTA-K0-06, DeepRTA-K0-10, DeepRTA-K0-16, DeepRTA-K0-18, and DeepRTA-K0-20 against DeepRTA is an objective indication of the inventive and non-obvious character of the disclosed intensity contextualization unit.

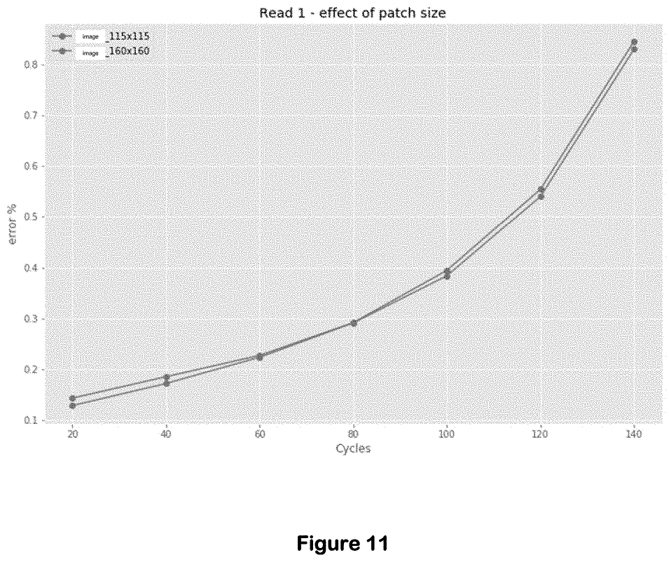

FIG. 11 shows base calling error rates when the disclosed neural network-based base caller configured with the disclosed intensity contextualization unit extracts intensity context data from an original input image of size 115×115 (red fitted line) versus an original input image of size 160×160 (blue fitted line). As demonstrated by FIG. 11, the base calling error rate is lower when intensity context data is gathered from a larger original input image of size 160×160.

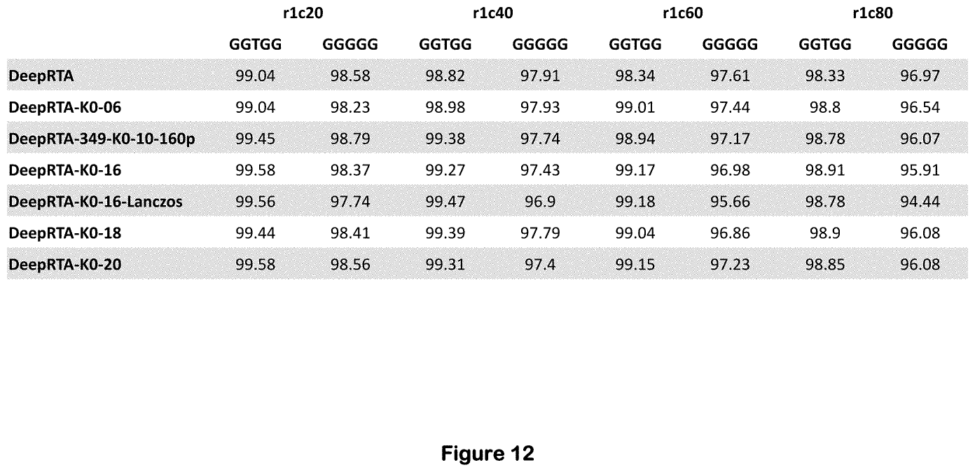

FIG. 12 shows base calling accuracy (1-base calling error rate) of the different configurations of the disclosed neural network-based base caller configured with the disclosed intensity contextualization unit, i.e., DeepRTA-K0-06, DeepRTA-349-K0-10-160p, DeepRTA-K0-16, DeepRTA-K0-16-Lanczos, DeepRTA-K0-18, and DeepRTA-K0-20 against DeepRTA over base calling homopolymers (e.g., GGGGG) and flanked-homopolymers (e.g., GGTGG).

As discussed above, in some implementations, the neural network-based base caller 124 makes a base call for a current sequencing cycle by processing a window of sequencing images for a plurality of sequencing cycles, including the current sequencing cycle contextualized by right and left sequencing cycles. Since the base “G” is indicated by a dark or off state in the sequencing images, repeat patterns of the base “G” can lead to erroneous base calls, particularly when the current sequencing cycle is for a non-G base (e.g., base “T”), but right and left flanked by Gs.

As demonstrated by FIG. 12, the different configurations of the disclosed neural network-based base caller configured with the disclosed intensity contextualization unit have a high base calling accuracy for such homopolymers (e.g., GGGGG) and flanked-homopolymers (e.g., GGTGG). One reason for this is that the disclosed intensity contextualization unit extracts intensity context beyond a given patch to inform the neural network-based base caller 124 that even though the flanking sequencing cycles represent the base “G”, the center sequencing cycle is a non-G base.

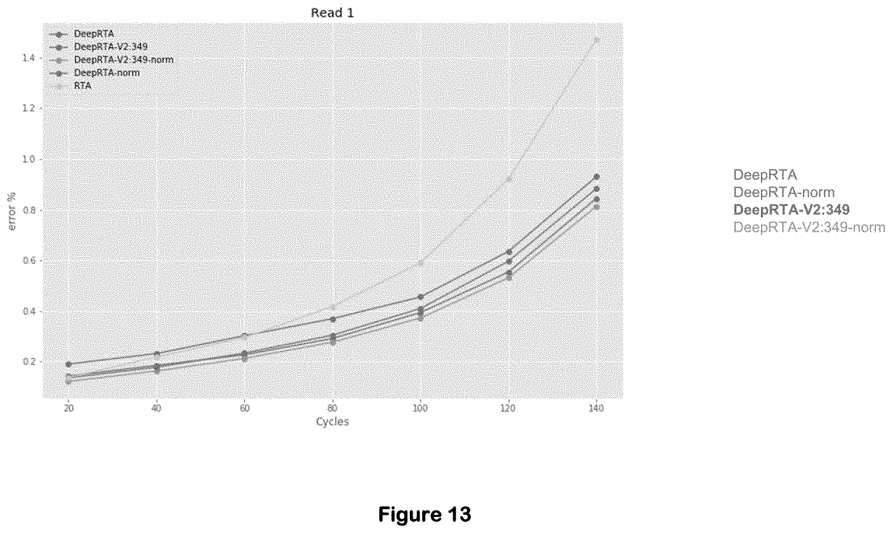

FIG. 13 compares base calling error rates of the disclosed neural network-based base caller configured with the disclosed intensity contextualization unit and trained on normalized sequencing images (“DeepRTA-V2:349”) against DeepRTA, RTA, the disclosed neural network-based base caller configured with the disclosed intensity contextualization unit, trained on, and performing inference on normalized sequencing images (“DeepRTA-V2:349”), and the DeepRTA trained on and performing inference on normalized sequencing images (“DeepRTA-norm”).

The normalized sequencing images are normalized to have a certain intensity distribution (e.g., they have intensity values in a lower percentile and a higher percentile (e.g., five percent of the normalized intensity values are below zero, another five percent of the normalized intensity values are greater than one, and the remaining ninety percent of the normalized intensity values are between zero and one)). Additional details and examples of normalization can be found in commonly owned U.S. Patent Application 62/979,384.

As demonstrated by FIG. 13, DeepRTA-V2:349 (blue fitted line) and DeepRTA-V2:349 (purple fitted line) outperform DeepRTA-norm (grey fitted line), DeepRTA (red fitted line), and RTA (orange fitted line).

### Sequencing System

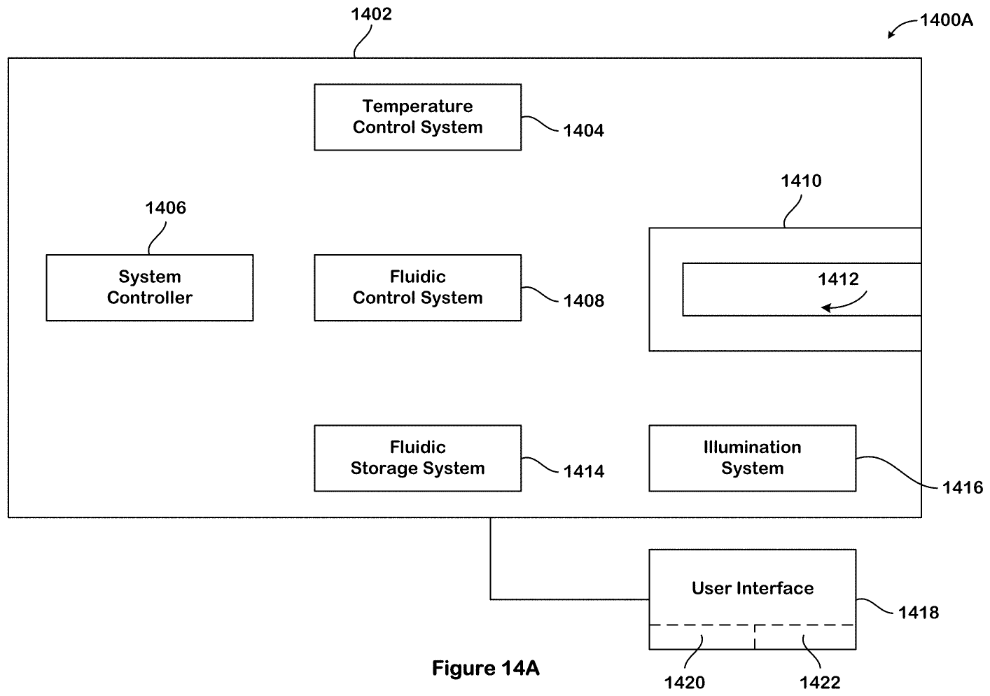

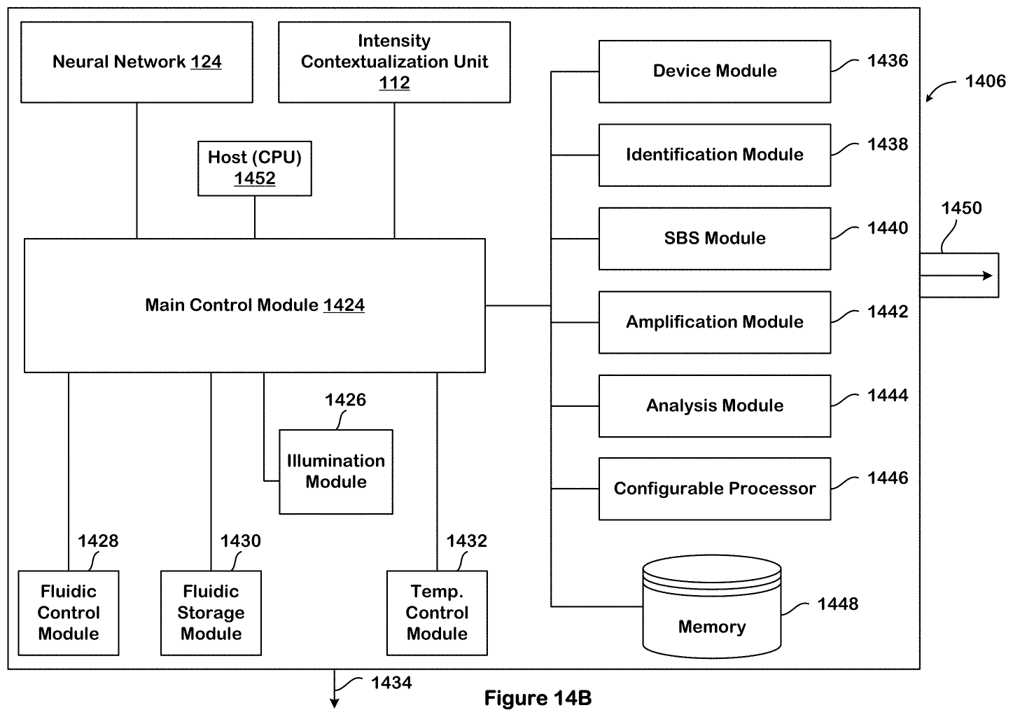

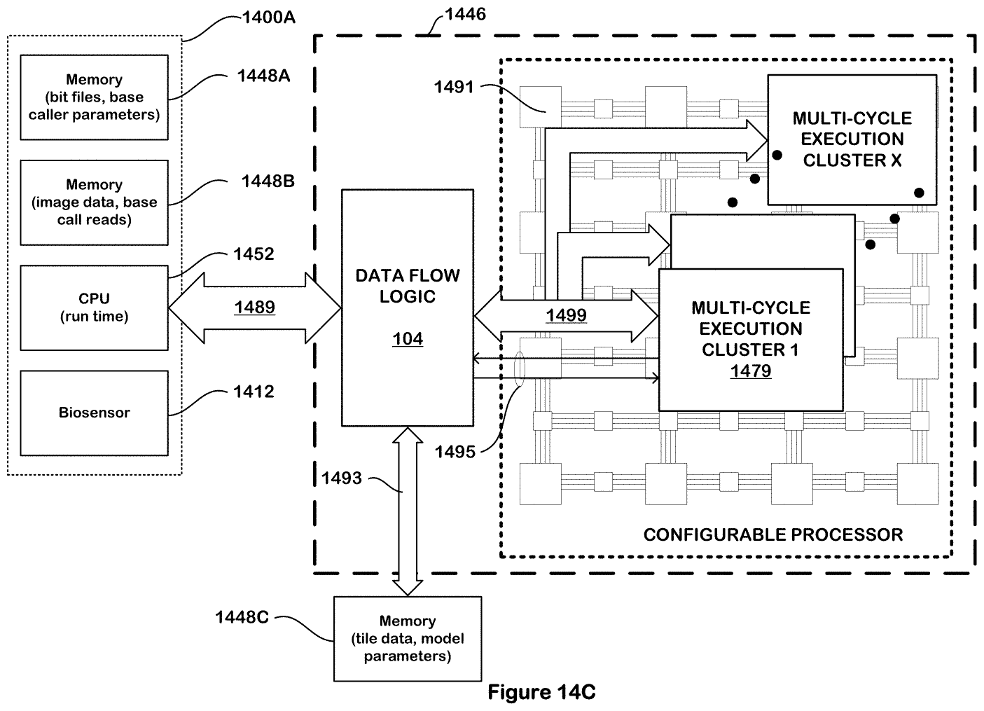

FIGS. 14A and 14B depict one implementation of a sequencing system 1400A. The sequencing system 1400A comprises a configurable processor 1446. The configurable processor 1446 implements the base calling techniques disclosed herein. The sequencing system is also referred to as a “sequencer.”

The sequencing system 1400A can operate to obtain any information or data that relates to at least one of a biological or chemical substance. In some implementations, the sequencing system 1400A is a workstation that may be similar to a bench-top device or desktop computer. For example, a majority (or all) of the systems and components for conducting the desired reactions can be within a common housing 1402.

In particular implementations, the sequencing system 1400A is a nucleic acid sequencing system configured for various applications, including but not limited to de novo sequencing, resequencing of whole genomes or target genomic regions, and metagenomics. The sequencer may also be used for DNA or RNA analysis. In some implementations, the sequencing system 1400A may also be configured to generate reaction sites in a biosensor. For example, the sequencing system 1400A may be configured to receive a sample and generate surface attached clusters of clonally amplified nucleic acids derived from the sample. Each cluster may constitute or be part of a reaction site in the biosensor.

The exemplary sequencing system 1400A may include a system receptacle or interface 1410 that is configured to interact with a biosensor 1412 to perform desired reactions within the biosensor 1412. In the following description with respect to FIG. 14A, the biosensor 1412 is loaded into the system receptacle 1410. However, it is understood that a cartridge that includes the biosensor 1412 may be inserted into the system receptacle 1410 and in some states the cartridge can be removed temporarily or permanently. As described above, the cartridge may include, among other things, fluidic control and fluidic storage components.

In particular implementations, the sequencing system 1400A is configured to perform a large number of parallel reactions within the biosensor 1412. The biosensor 1412 includes one or more reaction sites where desired reactions can occur. The reaction sites may be, for example, immobilized to a solid surface of the biosensor or immobilized to beads (or other movable substrates) that are located within corresponding reaction chambers of the biosensor. The reaction sites can include, for example, clusters of clonally amplified nucleic acids. The biosensor 1412 may include a solid-state imaging device (e.g., CCD or CMOS imager) and a flow cell mounted thereto. The flow cell may include one or more flow channels that receive a solution from the sequencing system 1400A and direct the solution toward the reaction sites. Optionally, the biosensor 1412 can be configured to engage a thermal element for transferring thermal energy into or out of the flow channel.

The sequencing system 1400A may include various components, assemblies, and systems (or sub-systems) that interact with each other to perform a predetermined method or assay protocol for biological or chemical analysis. For example, the sequencing system 1400A includes a system controller 1406 that may communicate with the various components, assemblies, and sub-systems of the sequencing system 1400A and also the biosensor 1412. For example, in addition to the system receptacle 1410, the sequencing system 1400A may also include a fluidic control system 1408 to control the flow of fluid throughout a fluid network of the sequencing system 1400A and the biosensor 1412; a fluid storage system 1414 that is configured to hold all fluids (e.g., gas or liquids) that may be used by the bioassay system; a temperature control system 1404 that may regulate the temperature of the fluid in the fluid network, the fluid storage system 1414, and/or the biosensor 1412; and an illumination system 1416 that is configured to illuminate the biosensor 1412. As described above, if a cartridge having the biosensor 1412 is loaded into the system receptacle 1410, the cartridge may also include fluidic control and fluidic storage components.

Also shown, the sequencing system 1400A may include a user interface 1418 that interacts with the user. For example, the user interface 1418 may include a display 1420 to display or request information from a user and a user input device 1422 to receive user inputs. In some implementations, the display 1420 and the user input device 1422 are the same device. For example, the user interface 1418 may include a touch-sensitive display configured to detect the presence of an individual's touch and also identify a location of the touch on the display. However, other user input devices 1422 may be used, such as a mouse, touchpad, keyboard, keypad, handheld scanner, voice-recognition system, motion-recognition system, and the like. As will be discussed in greater detail below, the sequencing system 1400A may communicate with various components, including the biosensor 1412 (e.g., in the form of a cartridge), to perform the desired reactions. The sequencing system 1400A may also be configured to analyze data obtained from the biosensor to provide a user with desired information.

The system controller 1406 may include any processor-based or microprocessor-based system, including systems using microcontrollers, reduced instruction set computers (RISC), application specific integrated circuits (ASICs), field programmable gate array (FPGAs), coarse-grained reconfigurable architectures (CGRAs), logic circuits, and any other circuit or processor capable of executing functions described herein. The above examples are exemplary only, and are thus not intended to limit in any way the definition and/or meaning of the term system controller. In the exemplary implementation, the system controller 1406 executes a set of instructions that are stored in one or more storage elements, memories, or modules in order to at least one of obtain and analyze detection data. Detection data can include a plurality of sequences of pixel signals, such that a sequence of pixel signals from each of the millions of sensors (or pixels) can be detected over many base calling cycles. Storage elements may be in the form of information sources or physical memory elements within the sequencing system 1400A.

The set of instructions may include various commands that instruct the sequencing system 1400A or biosensor 1412 to perform specific operations such as the methods and processes of the various implementations described herein. The set of instructions may be in the form of a software program, which may form part of a tangible, non-transitory computer readable medium or media. As used herein, the terms “software” and “firmware” are interchangeable and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

The software may be in various forms such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the sequencing system 1400A, processed in response to user inputs, or processed in response to a request made by another processing machine (e.g., a remote request through a communication link). In the illustrated implementation, the system controller 1406 includes an analysis module 1444. In other implementations, system controller 1406 does not include the analysis module 1444 and instead has access to the analysis module 1444 (e.g., the analysis module 1444 may be separately hosted on cloud).

The system controller 1406 may be connected to the biosensor 1412 and the other components of the sequencing system 1400A via communication links The system controller 1406 may also be communicatively connected to off-site systems or servers. The communication links may be hardwired, corded, or wireless. The system controller 1406 may receive user inputs or commands, from the user interface 1418 and the user input device 1422.

The fluidic control system 1408 includes a fluid network and is configured to direct and regulate the flow of one or more fluids through the fluid network. The fluid network may be in fluid communication with the biosensor 1412 and the fluid storage system 1414. For example, select fluids may be drawn from the fluid storage system 1414 and directed to the biosensor 1412 in a controlled manner, or the fluids may be drawn from the biosensor 1412 and directed toward, for example, a waste reservoir in the fluid storage system 1414. Although not shown, the fluidic control system 1408 may include flow sensors that detect a flow rate or pressure of the fluids within the fluid network. The sensors may communicate with the system controller 1406.

The temperature control system 1404 is configured to regulate the temperature of fluids at different regions of the fluid network, the fluid storage system 1414, and/or the biosensor 1412. For example, the temperature control system 1404 may include a thermocycler that interfaces with the biosensor 1412 and controls the temperature of the fluid that flows along the reaction sites in the biosensor 1412. The temperature control system 1404 may also regulate the temperature of solid elements or components of the sequencing system 1400A or the biosensor 1412. Although not shown, the temperature control system 1404 may include sensors to detect the temperature of the fluid or other components. The sensors may communicate with the system controller 1406.

The fluid storage system 1414 is in fluid communication with the biosensor 1412 and may store various reaction components or reactants that are used to conduct the desired reactions therein. The fluid storage system 1414 may also store fluids for washing or cleaning the fluid network and biosensor 1412 and for diluting the reactants. For example, the fluid storage system 1414 may include various reservoirs to store samples, reagents, enzymes, other biomolecules, buffer solutions, aqueous, and non-polar solutions, and the like. Furthermore, the fluid storage system 1414 may also include waste reservoirs for receiving waste products from the biosensor 1412. In implementations that include a cartridge, the cartridge may include one or more of a fluid storage system, fluidic control system or temperature control system. Accordingly, one or more of the components set forth herein as relating to those systems can be contained within a cartridge housing. For example, a cartridge can have various reservoirs to store samples, reagents, enzymes, other biomolecules, buffer solutions, aqueous, and non-polar solutions, waste, and the like. As such, one or more of a fluid storage system, fluidic control system or temperature control system can be removably engaged with a bioassay system via a cartridge or other biosensor.

The illumination system 1416 may include a light source (e.g., one or more LEDs) and a plurality of optical components to illuminate the biosensor. Examples of light sources may include lasers, arc lamps, LEDs, or laser diodes. The optical components may be, for example, reflectors, dichroics, beam splitters, collimators, lenses, filters, wedges, prisms, mirrors, detectors, and the like. In implementations that use an illumination system, the illumination system 1416 may be configured to direct an excitation light to reaction sites. As one example, fluorophores may be excited by green wavelengths of light, as such the wavelength of the excitation light may be approximately 1432 nm. In one implementation, the illumination system 1416 is configured to produce illumination that is parallel to a surface normal of a surface of the biosensor 1412. In another implementation, the illumination system 1416 is configured to produce illumination that is off-angle relative to the surface normal of the surface of the biosensor 1412. In yet another implementation, the illumination system 1416 is configured to produce illumination that has plural angles, including some parallel illumination and some off-angle illumination

The system receptacle or interface 1410 is configured to engage the biosensor 1412 in at least one of a mechanical, electrical, and fluidic manner. The system receptacle 1410 may hold the biosensor 1412 in a desired orientation to facilitate the flow of fluid through the biosensor 1412. The system receptacle 1410 may also include electrical contacts that are configured to engage the biosensor 1412 so that the sequencing system 1400A may communicate with the biosensor 1412 and/or provide power to the biosensor 1412. Furthermore, the system receptacle 1410 may include fluidic ports (e.g., nozzles) that are configured to engage the biosensor 1412. In some implementations, the biosensor 1412 is removably coupled to the system receptacle 1410 in a mechanical manner, in an electrical manner, and also in a fluidic manner.

In addition, the sequencing system 1400A may communicate remotely with other systems or networks or with other bioassay systems 1400A. Detection data obtained by the bioassay system(s) 1400A may be stored in a remote database.

FIG. 14B is a block diagram of a system controller 1406 that can be used in the system of FIG. 14A. In one implementation, the system controller 1406 includes one or more processors or modules that can communicate with one another. Each of the processors or modules may include an algorithm (e.g., instructions stored on a tangible and/or non-transitory computer readable storage medium) or sub-algorithms to perform particular processes. The system controller 1406 is illustrated conceptually as a collection of modules, but may be implemented utilizing any combination of dedicated hardware boards, DSPs, processors, etc. Alternatively, the system controller 1406 may be implemented utilizing an off-the-shelf PC with a single processor or multiple processors, with the functional operations distributed between the processors. As a further option, the modules described below may be implemented utilizing a hybrid configuration in which certain modular functions are performed utilizing dedicated hardware, while the remaining modular functions are performed utilizing an off-the-shelf PC and the like. The modules also may be implemented as software modules within a processing unit.

During operation, a communication port 1450 may transmit information (e.g., commands) to or receive information (e.g., data) from the biosensor 1412 (FIG. 14A) and/or the sub-systems 1408, 1414, 1404 (FIG. 14A). In implementations, the communication port 1450 may output a plurality of sequences of pixel signals. A communication link 1434 may receive user input from the user interface 1418 (FIG. 14A) and transmit data or information to the user interface 1418. Data from the biosensor 1412 or sub-systems 1408, 1414, 1404 may be processed by the system controller 1406 in real-time during a bioassay session. Additionally or alternatively, data may be stored temporarily in a system memory during a bioassay session and processed in slower than real-time or off-line operation.

As shown in FIG. 14B, the system controller 1406 may include a plurality of modules 1426-548 that communicate with a main control module 1424, along with a central processing unit (CPU) 1452. The main control module 1424 may communicate with the user interface 1418 (FIG. 14A). Although the modules 1426-548 are shown as communicating directly with the main control module 1424, the modules 1426-548 may also communicate directly with each other, the user interface 1418, and the biosensor 1412. Also, the modules 1426-548 may communicate with the main control module 1424 through the other modules.

The plurality of modules 1426-548 include system modules 1428-532, 1426 that communicate with the sub-systems 1408, 1414, 1404, and 1416, respectively. The fluidic control module 1428 may communicate with the fluidic control system 1408 to control the valves and flow sensors of the fluid network for controlling the flow of one or more fluids through the fluid network. The fluid storage module 1430 may notify the user when fluids are low or when the waste reservoir is at or near capacity. The fluid storage module 1430 may also communicate with the temperature control module 1432 so that the fluids may be stored at a desired temperature. The illumination module 1426 may communicate with the illumination system 1416 to illuminate the reaction sites at designated times during a protocol, such as after the desired reactions (e.g., binding events) have occurred. In some implementations, the illumination module 1426 may communicate with the illumination system 1416 to illuminate the reaction sites at designated angles.

The plurality of modules 1426-548 may also include a device module 1436 that communicates with the biosensor 1412 and an identification module 1438 that determines identification information relating to the biosensor 1412. The device module 1436 may, for example, communicate with the system receptacle 1410 to confirm that the biosensor has established an electrical and fluidic connection with the sequencing system 1400A. The identification module 1438 may receive signals that identify the biosensor 1412. The identification module 1438 may use the identity of the biosensor 1412 to provide other information to the user. For example, the identification module 1438 may determine and then display a lot number, a date of manufacture, or a protocol that is recommended to be run with the biosensor 1412.

The plurality of modules 1426-548 also includes an analysis module 1444 (also called signal processing module or signal processor) that receives and analyzes the signal data (e.g., image data) from the biosensor 1412. Analysis module 1444 includes memory (e.g., RAM or Flash) to store detection/image data. Detection data can include a plurality of sequences of pixel signals, such that a sequence of pixel signals from each of the millions of sensors (or pixels) can be detected over many base calling cycles. The signal data may be stored for subsequent analysis or may be transmitted to the user interface 1418 to display desired information to the user. In some implementations, the signal data may be processed by the solid-state imager (e.g., CMOS image sensor) before the analysis module 1444 receives the signal data.

The analysis module 1444 is configured to obtain image data from the light detectors at each of a plurality of sequencing cycles. The image data is derived from the emission signals detected by the light detectors and process the image data for each of the plurality of sequencing cycles through the neural network-based base caller 124 and produce a base call for at least some of the analytes at each of the plurality of sequencing cycle. The light detectors can be part of one or more over-head cameras (e g., Illumina's GAIIx's CCD camera taking images of the clusters on the biosensor 1412 from the top), or can be part of the biosensor 1412 itself (e g., Illumina's iSeq's CMOS image sensors underlying the clusters on the biosensor 1412 and taking images of the clusters from the bottom).

The output of the light detectors is the sequencing images, each depicting intensity emissions of the clusters and their surrounding background. The sequencing images depict intensity emissions generated as a result of nucleotide incorporation in the sequences during the sequencing. The intensity emissions are from associated analytes and their surrounding background. The sequencing images are stored in memory 1448.

Protocol modules 1440 and 1442 communicate with the main control module 1424 to control the operation of the sub-systems 1408, 1414, and 1404 when conducting predetermined assay protocols. The protocol modules 1440 and 1442 may include sets of instructions for instructing the sequencing system 1400A to perform specific operations pursuant to predetermined protocols. As shown, the protocol module may be a sequencing-by-synthesis (SBS) module 1440 that is configured to issue various commands for performing sequencing-by-synthesis processes. In SBS, extension of a nucleic acid primer along a nucleic acid template is monitored to determine the sequence of nucleotides in the template. The underlying chemical process can be polymerization (e.g., as catalyzed by a polymerase enzyme) or ligation (e.g., catalyzed by a ligase enzyme). In a particular polymerase-based SBS implementation, fluorescently labeled nucleotides are added to a primer (thereby extending the primer) in a template dependent fashion such that detection of the order and type of nucleotides added to the primer can be used to determine the sequence of the template. For example, to initiate a first SBS cycle, commands can be given to deliver one or more labeled nucleotides, DNA polymerase, etc., into/through a flow cell that houses an array of nucleic acid templates. The nucleic acid templates may be located at corresponding reaction sites. Those reaction sites where primer extension causes a labeled nucleotide to be incorporated can be detected through an imaging event. During an imaging event, the illumination system 1416 may provide an excitation light to the reaction sites. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for implementations that use reversible termination a command can be given to deliver a deblocking reagent to the flow cell (before or after detection occurs). One or more commands can be given to effect wash(es) between the various delivery steps. The cycle can then be repeated n times to extend the primer by n nucleotides, thereby detecting a sequence of length n. Exemplary sequencing techniques are described, for example, in Bentley et al., Nature 456:53-59 (2005); WO 04/015497; U.S. Pat. No. 7,057,026; WO 91/06675; WO 07/123744; U.S. Pat. Nos. 7,329,492; 7,211,414; 7,315,019; 7,405,251, and US 2005/014705052, each of which is incorporated herein by reference.

For the nucleotide delivery step of an SBS cycle, either a single type of nucleotide can be delivered at a time, or multiple different nucleotide types (e.g., A, C, T and G together) can be delivered. For a nucleotide delivery configuration where only a single type of nucleotide is present at a time, the different nucleotides need not have distinct labels since they can be distinguished based on temporal separation inherent in the individualized delivery. Accordingly, a sequencing method or apparatus can use single color detection. For example, an excitation source need only provide excitation at a single wavelength or in a single range of wavelengths. For a nucleotide delivery configuration where delivery results in multiple different nucleotides being present in the flow cell at one time, sites that incorporate different nucleotide types can be distinguished based on different fluorescent labels that are attached to respective nucleotide types in the mixture. For example, four different nucleotides can be used, each having one of four different fluorophores. In one implementation, the four different fluorophores can be distinguished using excitation in four different regions of the spectrum. For example, four different excitation radiation sources can be used. Alternatively, fewer than four different excitation sources can be used, but optical filtration of the excitation radiation from a single source can be used to produce different ranges of excitation radiation at the flow cell.

In some implementations, fewer than four different colors can be detected in a mixture having four different nucleotides. For example, pairs of nucleotides can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. Exemplary apparatus and methods for distinguishing four different nucleotides using detection of fewer than four colors are described for example in U.S. Pat. App. Ser. Nos. 61/535,294 and 61/619,575, which are incorporated herein by reference in their entireties. U.S. application Ser. No. 13/624,200, which was filed on Sep. 21, 2012, is also incorporated by reference in its entirety.

The plurality of protocol modules may also include a sample-preparation (or generation) module 1442 that is configured to issue commands to the fluidic control system 1408 and the temperature control system 1404 for amplifying a product within the biosensor 1412. For example, the biosensor 1412 may be engaged to the sequencing system 1400A. The amplification module 1442 may issue instructions to the fluidic control system 1408 to deliver necessary amplification components to reaction chambers within the biosensor 1412. In other implementations, the reaction sites may already contain some components for amplification, such as the template DNA and/or primers. After delivering the amplification components to the reaction chambers, the amplification module 1442 may instruct the temperature control system 1404 to cycle through different temperature stages according to known amplification protocols. In some implementations, the amplification and/or nucleotide incorporation is performed isothermally.

The SBS module 1440 may issue commands to perform bridge PCR where clusters of clonal amplicons are formed on localized areas within a channel of a flow cell. After generating the amplicons through bridge PCR, the amplicons may be “linearized” to make single stranded template DNA, or sstDNA, and a sequencing primer may be hybridized to a universal sequence that flanks a region of interest. For example, a reversible terminator-based sequencing by synthesis method can be used as set forth above or as follows.

Each base calling or sequencing cycle can extend an sstDNA by a single base which can be accomplished for example by using a modified DNA polymerase and a mixture of four types of nucleotides. The different types of nucleotides can have unique fluorescent labels, and each nucleotide can further have a reversible terminator that allows only a single-base incorporation to occur in each cycle. After a single base is added to the sstDNA, excitation light may be incident upon the reaction sites and fluorescent emissions may be detected. After detection, the fluorescent label and the terminator may be chemically cleaved from the sstDNA. Another similar base calling or sequencing cycle may follow. In such a sequencing protocol, the SBS module 1440 may instruct the fluidic control system 1408 to direct a flow of reagent and enzyme solutions through the biosensor 1412. Exemplary reversible terminator-based SBS methods which can be utilized with the apparatus and methods set forth herein are described in US Patent Application Publication No. 2007/0166705 A1, US Patent Application Publication No. 2006/0156*3901 A1, U.S. Pat. No. 7,057,026, US Patent Application Publication No. 2006/0240439 A1, US Patent Application Publication No. 2006/02514714709 A1, PCT Publication No. WO 05/065514, US Patent Application Publication No. 2005/014700900 A1, PCT Publication No. WO 06/05B199 and PCT Publication No. WO 07/01470251, each of which is incorporated herein by reference in its entirety. Exemplary reagents for reversible terminator-based SBS are described in 7,541,444; 7,057,026; 7,414,14716; 7,427,673; 7,566,537; 7,592,435 and WO 07/14535365, each of which is incorporated herein by reference in its entirety.

In some implementations, the amplification and SBS modules may operate in a single assay protocol where, for example, template nucleic acid is amplified and subsequently sequenced within the same cartridge.

The sequencing system 1400A may also allow the user to reconfigure an assay protocol. For example, the sequencing system 1400A may offer options to the user through the user interface 1418 for modifying the determined protocol. For example, if it is determined that the biosensor 1412 is to be used for amplification, the sequencing system 1400A may request a temperature for the annealing cycle. Furthermore, the sequencing system 1400A may issue warnings to a user if a user has provided user inputs that are generally not acceptable for the selected assay protocol.

In implementations, the biosensor 1412 includes millions of sensors (or pixels), each of which generates a plurality of sequences of pixel signals over successive base calling cycles. The analysis module 1444 detects the plurality of sequences of pixel signals and attributes them to corresponding sensors (or pixels) in accordance to the row-wise and/or column-wise location of the sensors on an array of sensors.

### Configurable Processor

FIG. 14C is a simplified block diagram of a system for analysis of sensor data from the sequencing system 1400A, such as base call sensor outputs. In the example of FIG. 14C, the system includes the configurable processor 1446. The configurable processor 1446 can execute a base caller (e.g., the neural network-based base caller 124) in coordination with a runtime program executed by the central processing unit (CPU) 1452 (i.e., a host processor). The sequencing system 1400A comprises the biosensor 1412 and flow cells. The flow cells can comprise one or more tiles in which clusters of genetic material are exposed to a sequence of analyte flows used to cause reactions in the clusters to identify the bases in the genetic material. The sensors sense the reactions for each cycle of the sequence in each tile of the flow cell to provide tile data. Genetic sequencing is a data intensive operation, which translates base call sensor data into sequences of base calls for each cluster of genetic material sensed in during a base call operation.

The system in this example includes the CPU 1452, which executes a runtime program to coordinate the base call operations, memory 1448B to store sequences of arrays of tile data, base call reads produced by the base calling operation, and other information used in the base call operations. Also, in this illustration the system includes memory 1448A to store a configuration file (or files), such as FPGA bit files, and model parameters for the neural networks used to configure and reconfigure the configurable processor 1446, and execute the neural networks. The sequencing system 1400A can include a program for configuring a configurable processor and in some implementations a reconfigurable processor to execute the neural networks.

The sequencing system 1400A is coupled by a bus 1489 to the configurable processor 1446. The bus 1489 can be implemented using a high throughput technology, such as in one example bus technology compatible with the PCIe standards (Peripheral Component Interconnect Express) currently maintained and developed by the PCI-SIG (PCI Special Interest Group). Also, in this example, a memory 1448A is coupled to the configurable processor 1446 by bus 1493. The memory 1448A can be on-board memory, disposed on a circuit board with the configurable processor 1446. The memory 1448A is used for high speed access by the configurable processor 1446 of working data used in the base call operation. The bus 1493 can also be implemented using a high throughput technology, such as bus technology compatible with the PCIe standards

Configurable processors, including field programmable gate arrays FPGAs, coarse grained reconfigurable arrays CGRAs, and other configurable and reconfigurable devices, can be configured to implement a variety of functions more efficiently or faster than might be achieved using a general purpose processor executing a computer program. Configuration of configurable processors involves compiling a functional description to produce a configuration file, referred to sometimes as a bitstream or bit file, and distributing the configuration file to the configurable elements on the processor. The configuration file defines the logic functions to be executed by the configurable processor, by configuring the circuit to set data flow patterns, use of distributed memory and other on-chip memory resources, lookup table contents, operations of configurable logic blocks and configurable execution units like multiply-and-accumulate units, configurable interconnects and other elements of the configurable array. A configurable processor is reconfigurable if the configuration file may be changed in the field, by changing the loaded configuration file. For example, the configuration file may be stored in volatile SRAM elements, in non-volatile read-write memory elements, and in combinations of the same, distributed among the array of configurable elements on the configurable or reconfigurable processor. A variety of commercially available configurable processors are suitable for use in a base calling operation as described herein. Examples include Google's Tensor Processing Unit (TPU)™, rackmount solutions like GX4 Rackmount Series™, GX9 Rackmount Series™, NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, Xilinx Alveo™ U200, Xilinx Alveo™ U250, Xilinx Alveo™ U280, Intel/Altera Stratix™ GX2800, Intel/Altera Stratix™ GX2800, and Intel StratixT™ GX10M. In some examples, a host CPU can be implemented on the same integrated circuit as the configurable processor.

Implementations described herein implement the neural network-based base caller 124 using the configurable processor 1446. The configuration file for the configurable processor 1446 can be implemented by specifying the logic functions to be executed using a high level description language HDL or a register transfer level RTL language specification. The specification can be compiled using the resources designed for the selected configurable processor to generate the configuration file. The same or similar specification can be compiled for the purposes of generating a design for an application-specific integrated circuit which may not be a configurable processor.

Alternatives for the configurable processor configurable processor 1446, in all implementations described herein, therefore include a configured processor comprising an application specific ASIC or special purpose integrated circuit or set of integrated circuits, or a system-on-a-chip SOC device, or a graphics processing unit (GPU) processor or a coarse-grained reconfigurable architecture (CGRA) processor, configured to execute a neural network based base call operation as described herein.

In general, configurable processors and configured processors described herein, as configured to execute runs of a neural network, are referred to herein as neural network processors.

The configurable processor 1446 is configured in this example by a configuration file loaded using a program executed by the CPU 1452, or by other sources, which configures the array of configurable elements 1491 (e.g., configuration logic blocks (CLB) such as look up tables (LUTs), flip-flops, compute processing units (PMUs), and compute memory units (CMUs), configurable I/O blocks, programmable interconnects), on the configurable processor to execute the base call function. In this example, the configuration includes data flow logic 104 which is coupled to the buses 1489 and 1493 and executes functions for distributing data and control parameters among the elements used in the base call operation.

Also, the configurable processor 1446 is configured with data flow logic 104 to execute the neural network-based base caller 124. The logic 104 comprises multi-cycle execution clusters (e.g., 1479) which, in this example, includes execution cluster 1 through execution cluster X. The number of multi-cycle execution clusters can be selected according to a trade-off involving the desired throughput of the operation, and the available resources on the configurable processor 1446.

The multi-cycle execution clusters are coupled to the data flow logic 104 by data flow paths 1499 implemented using configurable interconnect and memory resources on the configurable processor 1446. Also, the multi-cycle execution clusters are coupled to the data flow logic 104 by control paths 1495 implemented using configurable interconnect and memory resources for example on the configurable processor 1446, which provide control signals indicating available execution clusters, readiness to provide input units for execution of a run of the neural network-based base caller 124 to the available execution clusters, readiness to provide trained parameters for the neural network-based base caller 124, readiness to provide output patches of base call classification data, and other control data used for execution of the neural network-based base caller 124.

The configurable processor 1446 is configured to execute runs of the neural network-based base caller 124 using trained parameters to produce classification data for the sensing cycles of the base calling operation. A run of the neural network-based base caller 124 is executed to produce classification data for a subject sensing cycle of the base calling operation. A run of the neural network-based base caller 124 operates on a sequence including a number N of arrays of tile data from respective sensing cycles of N sensing cycles, where the N sensing cycles provide sensor data for different base call operations for one base position per operation in time sequence in the examples described herein. Optionally, some of the N sensing cycles can be out of sequence if needed according to a particular neural network model being executed. The number N can be any number greater than one. In some examples described herein, sensing cycles of the N sensing cycles represent a set of sensing cycles for at least one sensing cycle preceding the subject sensing cycle and at least one sensing cycle following the subject cycle in time sequence. Examples are described herein in which the number N is an integer equal to or greater than five.

The data flow logic 104 is configured to move tile data and at least some trained parameters of the model parameters from the memory 1448A to the configurable processor 1446 for runs of the neural network-based base caller 124, using input units for a given run including tile data for spatially aligned patches of the N arrays. The input units can be moved by direct memory access operations in one DMA operation, or in smaller units moved during available time slots in coordination with the execution of the neural network deployed.

Tile data for a sensing cycle as described herein can comprise an array of sensor data having one or more features. For example, the sensor data can comprise two images which are analyzed to identify one of four bases at a base position in a genetic sequence of DNA, RNA, or other genetic material. The tile data can also include metadata about the images and the sensors. For example, in implementations of the base calling operation, the tile data can comprise information about alignment of the images with the clusters such as distance from center information indicating the distance of each pixel in the array of sensor data from the center of a cluster of genetic material on the tile.

During execution of the neural network-based base caller 124 as described below, tile data can also include data produced during execution of the neural network-based base caller 124, referred to as intermediate data, which can be reused rather than recomputed during a run of the neural network-based base caller 124. For example, during execution of the neural network-based base caller 124, the data flow logic 104 can write intermediate data to the memory 1448A in place of the sensor data for a given patch of an array of tile data. Implementations like this are described in more detail below.

As illustrated, a system is described for analysis of base call sensor output, comprising memory (e.g., 1448A) accessible by the runtime program storing tile data including sensor data for a tile from sensing cycles of a base calling operation. Also, the system includes a neural network processor, such as configurable processor 1446 having access to the memory. The neural network processor is configured to execute runs of a neural network using trained parameters to produce classification data for sensing cycles. As described herein, a run of the neural network is operating on a sequence of N arrays of tile data from respective sensing cycles of N sensing cycles, including a subject cycle, to produce the classification data for the subject cycle. The data flow logic 908 is provided to move tile data and the trained parameters from the memory to the neural network processor for runs of the neural network using input units including data for spatially aligned patches of the N arrays from respective sensing cycles of N sensing cycles.

Also, a system is described in which the neural network processor has access to the memory, and includes a plurality of execution clusters, the execution clusters in the plurality of execution clusters configured to execute a neural network. The data flow logic 104 has access to the memory and to execution clusters in the plurality of execution clusters, to provide input units of tile data to available execution clusters in the plurality of execution clusters, the input units including a number N of spatially aligned patches of arrays of tile data from respective sensing cycles, including a subject sensing cycle, and to cause the execution clusters to apply the N spatially aligned patches to the neural network to produce output patches of classification data for the spatially aligned patch of the subject sensing cycle, where N is greater than 1.

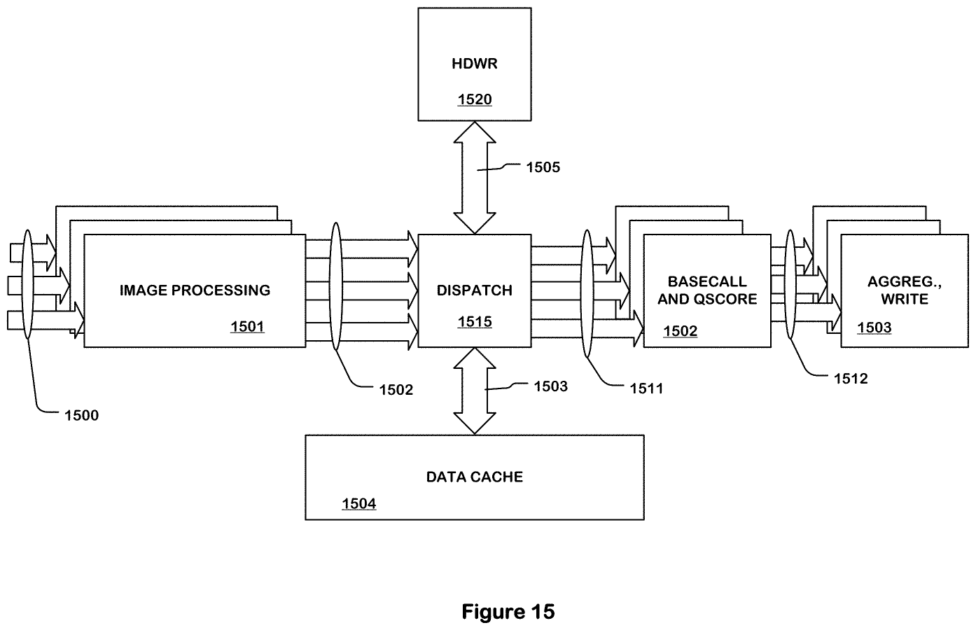

FIG. 15 is a simplified diagram showing aspects of the base calling operation, including functions of a runtime program executed by a host processor. In this diagram, the output of image sensors from a flow cell are provided on lines 1500 to image processing threads 1501, which can perform processes on images such as alignment and arrangement in an array of sensor data for the individual tiles and resampling of images, and can be used by processes which calculate a tile cluster mask for each tile in the flow cell, which identifies pixels in the array of sensor data that correspond to clusters of genetic material on the corresponding tile of the flow cell. The outputs of the image processing threads 1501 are provided on lines 1502 to a dispatch logic 1510 in the CPU which routes the arrays of tile data to a data cache 1504 (e.g., SSD storage) on a high-speed bus 1503, or on high-speed bus 1505 to the neural network processor hardware 1520, such as the configurable processor 1446 of FIG. 14C, according to the state of the base calling operation. The processed and transformed images can be stored on the data cache 1504 for sensing cycles that were previously used. The hardware 1520 returns classification data output by the neural network to the dispatch logic 1515, which passes the information to the data cache 1504, or on lines 1511 to threads 1502 that perform base call and quality score computations using the classification data, and can arrange the data in standard formats for base call reads. The outputs of the threads 1502 that perform base calling and quality score computations are provided on lines 1512 to threads 1503 that aggregate the base call reads, perform other operations such as data compression, and write the resulting base call outputs to specified destinations for utilization by the customers.

In some embodiments, the host can include threads (not shown) that perform final processing of the output of the hardware 1520 in support of the neural network. For example, the hardware 1520 can provide outputs of classification data from a final layer of the multi-cluster neural network. The host processor can execute an output activation function, such as a softmax function, over the classification data to configure the data for use by the base call and quality score threads 1502. Also, the host processor can execute input operations (not shown), such as batch normalization of the tile data prior to input to the hardware 1520.

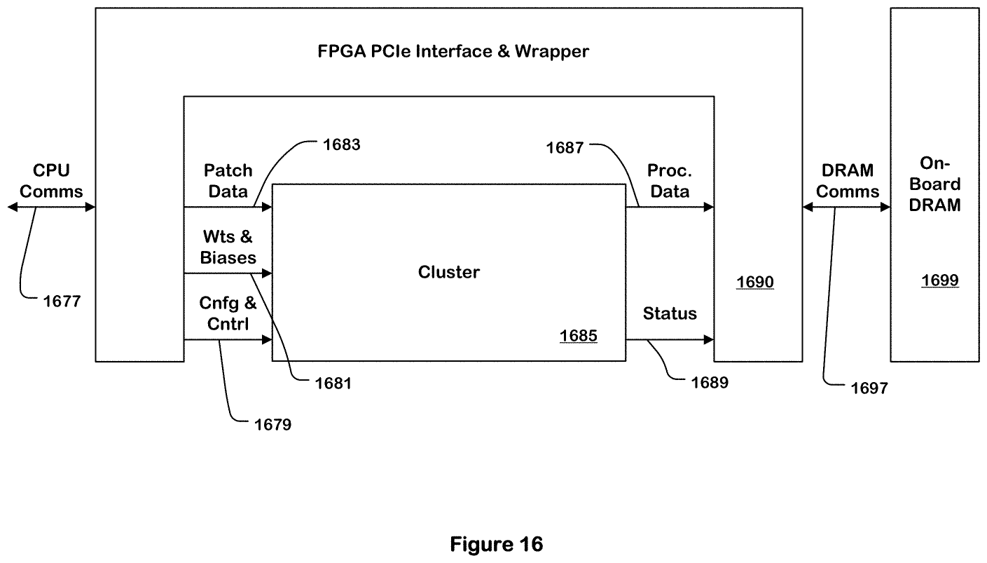

FIG. 16 is a simplified diagram of a configuration of a configurable processor 1446 such as that of FIG. 14C. In FIG. 16, the configurable processor 1446 comprises an FPGA with a plurality of high speed PCIe interfaces. The FPGA is configured with a wrapper 1690 which comprises the data flow logic 104 described with reference to FIG. 14C. The wrapper 1690 manages the interface and coordination with a runtime program in the CPU across the CPU communication link 1677 and manages communication with the on-board DRAM 1699 (e.g., memory 1448A) via DRAM communication link 1697. The data flow logic 104 in the wrapper 1690 provides patch data retrieved by traversing the arrays of tile data on the on-board DRAM 1699 for the number N cycles to a cluster 1685, and retrieves process data 1687 from the cluster 1685 for delivery back to the on-board DRAM 1699. The wrapper 1690 also manages transfer of data between the on-board DRAM 1699 and host memory, for both the input arrays of tile data, and for the output patches of classification data. The wrapper transfers patch data on line 1683 to the allocated cluster 1685. The wrapper provides trained parameters, such as weights and biases on line 1681 to the cluster 1685 retrieved from the on-board DRAM 1699. The wrapper provides configuration and control data on line 1679 to the cluster 1685 provided from, or generated in response to, the runtime program on the host via the CPU communication link 1677. The cluster can also provide status signals on line 1689 to the wrapper 1690, which are used in cooperation with control signals from the host to manage traversal of the arrays of tile data to provide spatially aligned patch data, and to execute the multi-cycle neural network over the patch data using the resources of the cluster 1685.

As mentioned above, there can be multiple clusters on a single configurable processor managed by the wrapper 1690 configured for executing on corresponding ones of multiple patches of the tile data. Each cluster can be configured to provide classification data for base calls in a subject sensing cycle using the tile data of multiple sensing cycles described herein.

In examples of the system, model data, including kernel data like filter weights and biases can be sent from the host CPU to the configurable processor, so that the model can be updated as a function of cycle number. A base calling operation can comprise, for a representative example, on the order of hundreds of sensing cycles. Base calling operation can include paired end reads in some embodiments. For example, the model trained parameters may be updated once every 20 cycles (or other number of cycles), or according to update patterns implemented for particular systems and neural network models. In some embodiments including paired end reads in which a sequence for a given string in a genetic cluster on a tile includes a first part extending from a first end down (or up) the string, and a second part extending from a second end up (or down) the string, the trained parameters can be updated on the transition from the first part to the second part.

In some examples, image data for multiple cycles of sensing data for a tile can be sent from the CPU to the wrapper 1690. The wrapper 1690 can optionally do some pre-processing and transformation of the sensing data and write the information to the on-board DRAM 1699. The input tile data for each sensing cycle can include arrays of sensor data including on the order of 4000×3000 pixels per sensing cycle per tile or more, with two features representing colors of two images of the tile, and one or two bytes per feature per pixel. For an embodiment in which the number N is three sensing cycles to be used in each run of the multi-cycle neural network, the array of tile data for each run of the multi-cycle neural network can consume on the order of hundreds of megabytes per tile. In some embodiments of the system, the tile data also includes an array of DFC data, stored once per tile, or other type of metadata about the sensor data and the tiles.

In operation, when a multi-cycle cluster is available, the wrapper allocates a patch to the cluster. The wrapper fetches a next patch of tile data in the traversal of the tile and sends it to the allocated cluster along with appropriate control and configuration information. The cluster can be configured with enough memory on the configurable processor to hold a patch of data including patches from multiple cycles in some systems, that is being worked on in place, and a patch of data that is to be worked on when the current patch of processing is finished using a ping-pong buffer technique or raster scanning technique in various embodiments.

When an allocated cluster completes its run of the neural network for the current patch and produces an output patch, it will signal the wrapper. The wrapper will read the output patch from the allocated cluster, or alternatively the allocated cluster will push the data out to the wrapper. Then the wrapper will assemble output patches for the processed tile in the DRAM 1699. When the processing of the entire tile has been completed, and the output patches of data transferred to the DRAM, the wrapper sends the processed output array for the tile back to the host/CPU in a specified format. In some embodiments, the on-board DRAM 1699 is managed by memory management logic in the wrapper 1690. The runtime program can control the sequencing operations to complete analysis of all the arrays of tile data for all the cycles in the run in a continuous flow to provide real time analysis.

### Computer System

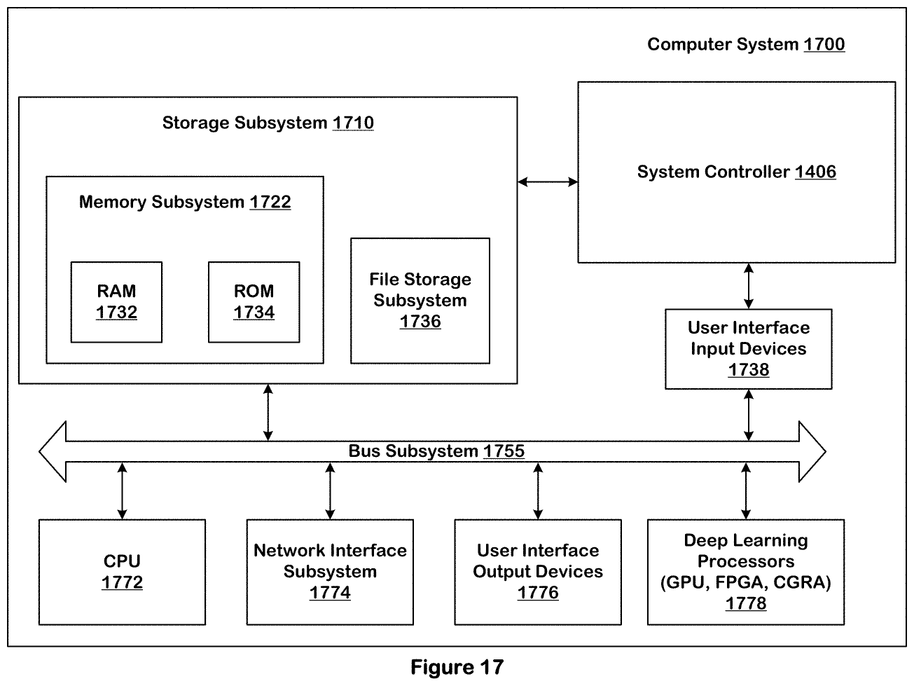

FIG. 17 is a computer system 1700 that can be used by the sequencing system 500A to implement the base calling techniques disclosed herein. Computer system 1700 includes at least one central processing unit (CPU) 1772 that communicates with a number of peripheral devices via bus subsystem 1755. These peripheral devices can include a storage subsystem 858 including, for example, memory devices and a file storage subsystem 1736, user interface input devices 1738, user interface output devices 1776, and a network interface subsystem 1774. The input and output devices allow user interaction with computer system 1700. Network interface subsystem 1774 provides an interface to outside networks, including an interface to corresponding interface devices in other computer systems.

In one implementation, the system controller 1406 is communicably linked to the storage subsystem 1710 and the user interface input devices 1738.

User interface input devices 1738 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1700.

User interface output devices 1776 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1700 to the user or to another machine or computer system.

Storage subsystem 858 stores programming and data constructs that provide the functionality of some or all of the modules and methods described herein. These software modules are generally executed by deep learning processors 1778.

Deep learning processors 1778 can be graphics processing units (GPUs), field-programmable gate arrays (FPGAs), application-specific integrated circuits (ASICs), and/or coarse-grained reconfigurable architectures (CGRAs). Deep learning processors 1778 can be hosted by a deep learning cloud platform such as Google Cloud Platform™, Xilinx™, and Cirrascale™. Examples of deep learning processors 1778 include Google's Tensor Processing Unit (TPU)™, rackmount solutions like GX4 Rackmount Series™, GX17 Rackmount Series™, NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 1722 used in the storage subsystem 858 can include a number of memories including a main random access memory (RAM) 1732 for storage of instructions and data during program execution and a read only memory (ROM) 1734 in which fixed instructions are stored. A file storage subsystem 1736 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1736 in the storage subsystem 858, or in other machines accessible by the processor.

Bus subsystem 1755 provides a mechanism for letting the various components and subsystems of computer system 1700 communicate with each other as intended. Although bus subsystem 1755 is shown schematically as a single bus, alternative implementations of the bus subsystem can use multiple busses.

Computer system 1700 itself can be of varying types including a personal computer, a portable computer, a workstation, a computer terminal, a network computer, a television, a mainframe, a server farm, a widely-distributed set of loosely networked computers, or any other data processing system or user device. Due to the ever changing nature of computers and networks, the description of computer system 1700 depicted in FIG. 17 is intended only as a specific example for purposes of illustrating the preferred implementations of the present invention. Many other configurations of computer system 1700 are possible having more or less components than the computer system depicted in FIG. 17.

### Particular Implementations

The technology disclosed provides an artificial intelligence-based base caller with contextual awareness. The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

Various processes and steps of the methods set forth herein can be carried out using a computer. The computer can include a processor that is part of a detection device, networked with a detection device used to obtain the data that is processed by the computer or separate from the detection device. In some implementations, information (e.g., image data) may be transmitted between components of a system disclosed herein directly or via a computer network. A local area network (LAN) or wide area network (WAN) may be a corporate computing network, including access to the Internet, to which computers and computing devices comprising the system are connected. In one implementation, the LAN conforms to the transmission control protocol/internet protocol (TCP/IP) industry standard In some instances, the information (e.g., image data) is input to a system disclosed herein via an input device (e.g., disk drive, compact disk player, USB port etc.). In some instances, the information is received by loading the information, e.g., from a storage device such as a disk or flash drive.

A processor that is used to run an algorithm or other process set forth herein may comprise a microprocessor. The microprocessor may be any conventional general purpose single- or multi-chip microprocessor such as a Pentium™ processor made by Intel Corporation. A particularly useful computer can utilize an Intel Ivybridge dual-12 core processor, LSI raid controller, having 128 GB of RAM, and 2 TB solid state disk drive. In addition, the processor may comprise any conventional special purpose processor such as a digital signal processor or a graphics processor. The processor typically has conventional address lines, conventional data lines, and one or more conventional control lines.

The implementations disclosed herein may be implemented as a method, apparatus, system or article of manufacture using standard programming or engineering techniques to produce software, firmware, hardware, or any combination thereof. The term “article of manufacture” as used herein refers to code or logic implemented in hardware or computer readable media such as optical storage devices, and volatile or non-volatile memory devices. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices. In particular implementations, information or algorithms set forth herein are present in non-transient storage media.

In particular implementations, a computer-implemented method set forth herein can occur in real time while multiple images of an object are being obtained. Such real time analysis is particularly useful for nucleic acid sequencing applications wherein an array of nucleic acids is subjected to repeated cycles of fluidic and detection steps. Analysis of the sequencing data can often be computationally intensive such that it can be beneficial to perform the methods set forth herein in real time or in the background while other data acquisition or analysis algorithms are in process. Example real time analysis methods that can be used with the present methods are those used for the MiSeq and HiSeq sequencing devices commercially available from Illumina, Inc. (San Diego, Calif.) and/or described in US Pat. App. Pub. No. 2012/0020537 A1, which is incorporated herein by reference.

We disclose a system for base calling. The system includes memory, a data flow logic, a neural network, and an intensity contextualization unit.

The memory stores images that depict intensity emissions of a set of analytes. The intensity emissions are generated by analytes in the set of analytes during sequencing cycles of a sequencing run. The images have the intensity values for one or more intensity channels.

The data flow logic has access to the memory and is configured to provide a neural network access to the images on a patch-by-patch basis. The patches in an image depict the intensity emissions for a subset of the analytes. The patches have undiverse intensity patterns due to limited base diversity of analytes in the subset.

The neural network has a plurality of convolution filters. Convolution filters in the plurality of convolution filters have receptive fields confined to the patches. The convolution filters are configured to detect intensity patterns in the patches with losses in detection due to the undiverse intensity patterns and the confined receptive fields.

The intensity contextualization unit is configured to determine intensity context data based on intensity values in the images and store the intensity context data in the memory.

The data flow logic is configured to append the intensity context data to the patches to generate intensity contextualized images and provide the intensity contextualized images to the neural network.

The neural network is configured to apply the convolution filters on the intensity contextualized images and generates base call classifications. The intensity context data in the intensity contextualized images compensates for the losses in detection.

The system described in this section and other sections of the technology disclosed can include one or more of the following features and/or features described in connection with additional systems disclosed. In the interest of conciseness, the combinations of features disclosed in this application are not individually enumerated and are not repeated with each base set of features. The reader will understand how features identified in this system can readily be combined with sets of base features identified as implementations in other sections of this application.

The intensity context data specifies summary statistics of the intensity values. In one implementation, the intensity context data identifies a maximum value in the intensity values. In one implementation, the intensity context data identifies a minimum value in the intensity values.

In one implementation, the intensity context data identifies a mean of the intensity values. In one implementation, the intensity context data identifies a mode of the intensity values. In one implementation, the intensity context data identifies a standard deviation of the intensity values. In one implementation, the intensity context data identifies a variance of the intensity values.

In one implementation, the intensity context data identifies a skewness of the intensity values. In one implementation, the intensity context data identifies a kurtosis of the intensity values. In one implementation, the intensity context data identifies an entropy of the intensity values.

In one implementation, the intensity context data identifies one or more percentiles of the intensity values. In one implementation, the intensity context data identifies a delta between at least one of the maximum value and the minimum value, the maximum value and the mean, the mean and the minimum value, and a higher one of the percentiles and a lower one of the percentiles. In one implementation, the intensity context data identifies a sum of the intensity values.

In one implementation, the intensity contextualization unit determines a plurality of maximum values by dividing the intensity values into groups and determining a maximum value for each of the groups. The intensity context data identifies the smallest value in the plurality of maximum values.

In one implementation, the intensity contextualization unit determines a plurality of minimum values by dividing the intensity values into groups and determining a minimum value for each of the groups. The intensity context data identifies the largest value in the plurality of minimum values.

In one implementation, the intensity contextualization unit determines a plurality of sums by dividing the intensity values into groups and determining a sum of intensity values in each of the groups. The intensity context data identifies the smallest value in the plurality of sums. In other implementations, the intensity context data identifies the largest value in the plurality of sums. In yet other implementations, the intensity context data identifies a mean of the plurality of sums.

In one implementation, the intensity contextualization unit has a plurality of convolution pipelines. Each of the convolution pipelines has a plurality of convolution filters. Convolution filters in the plurality of convolution filters have varying filter sizes. The convolution filters have varying filter strides.

In one implementation, each of the convolution pipelines processes an image to generate a plurality of convolved representations of the image.

In one implementation, the intensity context data has a context channel for each convolved representation in the plurality of convolved representations. The context channel has as many concatenated copies of a respective one of the convolved representations as required to match a size of the image. In some implementations, each convolved representation is of size 1×1. The concatenated copies are pixelwise appended to the image.

We disclose a computer-implemented method of base calling. The method includes accessing images that depict intensity emissions of a set of analytes. The intensity emissions are generated by analytes in the set of analytes during sequencing cycles of a sequencing run. The method includes processing the images on a patch-by-patch basis, and thereby generating patches. The patches depict the intensity emissions for a subset of the analytes. The method includes determining intensity context data based on intensity values in the images. The method includes appending the intensity context data to the patches and generating intensity contextualized images. The method includes processing the intensity contextualized images and generating base call classifications.

Other implementations of the method described in this section can include a non-transitory computer readable storage medium storing instructions executable by a processor to perform any of the methods described above. Yet another implementation of the method described in this section can include a system including memory and one or more processors operable to execute instructions, stored in the memory, to perform any of the methods described above.

In one implementation, a self-normalizing neural network is disclosed. The self-normalizing neural network comprises a normalization layer (e.g., the intensity contextualization unit 112). The normalization layer is configured to determine one or more normalization parameters from an input on an input-by-input basis. The normalization layer is further configured to append context data characterizing the normalization parameters to patches accessed from the input. Consider, for example, two inputs, such as two images. Then, the normalization layer determines a first set of normalization parameters for the first image and a second set of normalization parameters for the second image. This is different from other normalization techniques like batch normalization, which learns a fixed set of normalization parameters and uses them for a whole batch of inputs. In contrast, the normalization parameters determined by the disclosed normalization layer are specific to a given input, and determined at runtime (e.g., at inference). During training, the normalization layer is trained to generate normalization parameters that are specific to a subject input.

The self-normalizing neural network further comprises runtime logic. The runtime logic is configured to process the patches appended with the context data through the self-normalizing neural network to generate an output.

In one implementation, the normalization layer is further configured to determine respective normalization parameters for respective inputs at runtime. In another implementation, the normalization parameters are summary statistics about intensity values in the input.

In one implementation, the context data includes the summary statistics in a pixel-wise encoding. In one implementation, the context data is pixel-wise encoded to the patches.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims

What is claimed is:

1. A system for base calling, comprising:
memory storing images that depict intensity emissions of a set of analytes, the intensity emissions generated by analytes in the set of analytes during sequencing cycles of a sequencing run;
data flow logic having access to the memory and configured to provide a neural network access to the images on a patch-by-patch basis, patches in an image depicting the intensity emissions for a subset of the analytes, and the patches having undiverse intensity patterns due to limited base diversity of analytes in the subset;
the neural network with a plurality of convolution filters, convolution filters in the plurality of convolution filters having receptive fields confined to the patches, and the convolution filters configured to detect intensity patterns in the patches with losses in detection due to the undiverse intensity patterns and the confined receptive fields;
an intensity contextualization unit configured to determine intensity context data based on intensity values in the images and store the intensity context data in the memory;
the data flow logic configured to append the intensity context data to the patches to generate intensity contextualized images and provide the intensity contextualized images to the neural network; and
the neural network configured to apply the convolution filters on the intensity contextualized images and generate base call classifications, the intensity context data in the intensity contextualized images compensating for the losses in detection.

2. The system of claim 1, wherein the images have the intensity values for one or more intensity channels.

3. The system of claim 2, wherein the intensity context data specifies summary statistics of the intensity values.

4. The system of claim 3, wherein the intensity context data identifies a maximum value in the intensity values.

5. The system of claim 4, wherein the intensity context data identifies a minimum value in the intensity values.

6. The system of claim 5, wherein the intensity context data identifies a mean of the intensity values.

7. The system of claim 6, wherein the intensity context data identifies a mode of the intensity values.

8. The system of claim 7, wherein the intensity context data identifies a standard deviation of the intensity values.

9. The system of claim 8, wherein the intensity context data identifies a variance of the intensity values.

10. The system of claim 9, wherein the intensity context data identifies a skewness of the intensity values.

11. The system of claim 10, wherein the intensity context data identifies a kurtosis of the intensity values.

12. The system of claim 11, wherein the intensity context data identifies an entropy of the intensity values.

13. The system of claim 12, wherein the intensity context data identifies one or more percentiles of the intensity values.

14. The system of claim 13, wherein the intensity context data identifies a delta between at least one of the maximum value and the minimum value, the maximum value and the mean, the mean and the minimum value, and a higher one of the percentiles and a lower one of the percentiles.

15. The system of claim 14, wherein the intensity context data identifies a sum of the intensity values.

16. The system of claim 15, wherein the intensity contextualization unit determines a plurality of maximum values by dividing the intensity values into groups and determining a maximum value for each of the groups, and wherein the intensity context data identifies the smallest value in the plurality of maximum values.

17. The system of claim 16, wherein the intensity contextualization unit determines a plurality of minimum values by dividing the intensity values into groups and determining a minimum value for each of the groups, wherein the intensity context data identifies the largest value in the plurality of minimum values.

18. The system of claim 17, wherein the intensity contextualization unit determines a plurality of sums by dividing the intensity values into groups and determining a sum of intensity values in each of the groups, wherein the intensity context data identifies the smallest value in the plurality of sums.

19. The system of claim 18, wherein the intensity context data identifies the largest value in the plurality of sums.

20. The system of claim 19, wherein the intensity context data identifies a mean of the plurality of sums.

21. The system of claim 20, wherein the intensity contextualization unit has a plurality of convolution pipelines, wherein each of the convolution pipelines has a plurality of convolution filters, wherein convolution filters in the plurality of convolution filters have varying filter sizes, and wherein the convolution filters have varying filter strides.

22. The system of claim 21, wherein each of the convolution pipelines processes an image to generate a plurality of convolved representations of the image.

23. The system of claim 22, wherein the intensity context data has a context channel for each convolved representation in the plurality of convolved representations, wherein the context channel has as many concatenated copies of a respective one of the convolved representations as required to match a size of the image.

24. The system of claim 23, wherein each convolved representation is of size 1×1, wherein the concatenated copies are pixelwise appended to the image.

25. A computer-implemented method of base calling, including:
accessing images that depict intensity emissions of a set of analytes, the intensity emissions generated by analytes in the set of analytes during sequencing cycles of a sequencing run;
processing the images on a patch-by-patch basis to generate patches, the patches depicting the intensity emissions for a subset of the analytes;
determining intensity context data based on intensity values in the images;
appending the intensity context data to the patches and genemting intensity contextualized images; and
processing the intensity contextualized images and generating base call classifications.

26. A system including one or more processors coupled to memory, the memory loaded with computer instructions to perform base calling, the instructions, when executed on the processors, implement actions comprising:
accessing images that depict intensity emissions of a set of analytes, the intensity emissions generated by analytes in the set of analytes during sequencing cycles of a sequencing run;
processing the images on a patch-by-patch basis to generate patches, the patches depicting the intensity emissions for a subset of the analytes;
determining intensity context data based on intensity values in the images;
appending the intensity context data to the patches and genemting intensity contextualized images; and
processing the intensity contextualized images and generating base call classifications.

27. A non-transitory computer readable storage medium impressed with computer program instructions for base calling, the instructions, when executed on a processor, implement a method comprising:
accessing images that depict intensity emissions of a set of analytes, the intensity emissions generated by analytes in the set of analytes during sequencing cycles of a sequencing run;
processing the images on a patch-by-patch basis to generate patches, the patches depicting the intensity emissions for a subset of the analytes;
determining intensity context data based on intensity values in the images;
appending the intensity context data to the patches and generating intensity contextualized images; and
processing the intensity contextualized images and generating base call classifications.

28. A self-normalizing neural network, comprising:
a normalization layer configured to determine one or more normalization parameters from an input on an input-by-input basis, and append context data characterizing the normalization parameters to patches accessed from the input; and
runtime logic configured to process the patches appended with the context data through the self-normalizing neural network to generate an output.

29. The self-normalizing neural network of claim 28, wherein the normalization layer is further configured to determine respective normalization parameters for respective inputs at runtime.

30. The self-normalizing neural network of claim 28, wherein the normalization parameters are summary statistics about intensity values in the input.

31. The self-normalizing neural network of claim 30, wherein the context data includes the summary statistics in a pixel-wise encoding.

32. The self-normalizing neural network of claim 31, wherein the context data is pixel-wise encoded to the patches.
